(19) United States
(12) Patent Application Publication
(10) Pub. No.: US 2013/0150685 A1
(43) Pub. Date: Jun. 13, 2013

(54) SYSTEM AND METHOD FOR MONITORING A SURGICAL SITE

(71) Applicant: Landy Aaron Toth, Newtown, PA (US)

(72) Inventor: Landy Aaron Toth, Newtown, PA (US)

(21) Appl. No.: 13/817,862

(22) PCT Filed: Aug. 23, 2011
(86) PCT No.: PCT/US11/48736

§ 371 Date: Feb. 20, 2013

Related U.S. Application Data
(60) Provisional application No. 61/376,254, filed on Aug. 23, 2010.

(51) Int. Cl. A61B 5/07

(52) U.S. Cl. CPC A61B 5/073; A61B 5/07
USPC 600/302

(57) ABSTRACT

A system and method for monitoring a surgical site within a body, including an elongate sensory subsystem adapted to be placed or implanted near to the surgical site including a sensor for collecting physiological data from the surgical site and a communication subsystem in communication with the elongate sensory subsystem. An elongate probe extending from the communication subsystem to the vicinity of the surgical site adapted to collect physiological data pertaining to the surgical site. A method for monitoring the surgical site to determine the early onset of complications is disclosed. A method for monitoring the integrity of an organ and in particular monitoring the integrity of an anastomosis is also disclosed.

### CROSS-REFERENCE TO RELATED APPLICATION

The present application is an international application which claims the benefit of and priority to U.S. Provisional Application Ser. No. 61/376,254 filed on Aug. 23, 2010, entitled “SYSTEM AND METHOD FOR MONITORING A SURGICAL SITE”, by Landy Toth, the entire contents of which is hereby incorporated by reference herein for all purposes

### BACKGROUND

1. Technical Field

The present disclosure is directed to systems and methods for monitoring a surgical site inside a body and more particularly to systems and methods for monitoring an anastomosis after surgery. The disclosure further provides systems and methods for monitoring the integrity of an organ inside a body. The disclosure further provides systems and methods for minimally invasively monitoring the health of an organ, transplant, tissue/implant interface and/or the vascular supply thereto inside a body.

2. Background

Over 46 million inpatient surgical procedures were performed in the United States in 2009. Many of these surgical procedures involved internal surgery on an organ, transplantation of an organ, or the implantation of a medical device. Needless to say, during the post-surgical recovery period, patient health, care and quick recovery are paramount to the successful outcome of such procedures.

At the same time there is also a need to lower healthcare expenditures while simultaneously improving patient recovery times in the post surgical setting.

One aspect of ensuring a quick and reliable recovery is to uncover post-surgical complications early so that they can be acted upon before an emergency situation arises. Early prediction of such complications often requires assessment of the surgical site.

As the surgical sites for many of these procedures are internal to the body, they can be particularly challenging to assess in practice. Methods such as laparoscopic inspection, angiography, Doppler ultrasonography, magnetic resonant imaging, laser Doppler flowmetry, optical transmission methods, laser and light spectroscopy, often require planned intervention, can be highly invasive, may lack sensitivity to clinically relevant indicators of the progression of the surgical site, can require moving of the patient, and often only assess the surgical site at the moment of observation or sampling.

Systemic methods for detecting complications at the surgical site often lack sensitivity as relevant symptoms often present only after significant damage to the surgical site or surrounding organs has occurred. Furthermore, systemic methods for detecting complications often provide single samples in time which can provide limited trending data relevant to the progression of the state of the surgical site.

In many cases, it is valuable to monitor tissue viability in the vicinity of a surgical site. Tissue parameters that maybe relevant in assessing viability within and in close vicinity to a surgical site include perfusion, ischemia, oxygenation, temperature, and edema among others. Other critical factors at a surgical site may include tissue separation, often caused by trauma, and accompanied by infection and/or immune response. Furthermore, intra-abdominal pressure can often be an early indicator of the onset of complications at an internal surgical site.

In the case of an anastomosis, in addition to monitoring tissue integrity, there is also a need to watch for clinically relevant complications such as abscess formation, onset of infection, wall thinning, and/or development of a leak. A leaking anastomosis can be a life threatening post surgical complication and early detection or even preemptive prediction of the onset of this condition can vastly improve survival rates associated with this complication. Early detection may allow for more minimally invasive corrective surgeries to be performed, allow for planned surgeries as opposed to emergency surgeries, and in the case of a gastrointestinal anastomosis, is less likely to be accompanied by significant amounts of foreign matter in the peritoneal cavity.

Thus there is a need for more convenient continuous monitoring of a surgical site during the post surgical recovery period.

In particular there is a need for early real-time predictors of complications associated with wound progression at a surgical site.

There is a need for minimally invasive continuous monitoring of surgical sites during the recovery period.

In addition, there is a need for minimally invasive real-time monitoring of an anastomosis and in particular a gastrointestinal anastomosis.

Furthermore, there is a need to continuously monitor the surgical site immediately following surgery through to full patient recovery. In particular there is a need for continued monitoring of the surgical site after the patient has started moving again as well as for prolonged periods after the patient has left the acute or intensive care wards of the hospital.

### SUMMARY

One objective of this disclosure is to provide a system and method for monitoring biological tissue at a tissue site, e.g., surgical site, inside a living body, e.g., mammalian body such as without limitation, a human body. Another objective is to provide a system and method for continuously monitoring such tissue. In one aspect, the present disclosure provides a simple, compact, and flexible self-powered system adapted to monitor tissue sites within a mammalian body, and communicating data relating to the monitored site to an outside device, e.g., a handheld device, smart phone, belt-worn device, and so forth. The disclosed system may be utilized without user intervention, thus enabling a surgeon to un-package a disposable portion of the device during surgery and place it at the site of interest. The system then operates autonomously to alert a nurse, doctor, or other clinician if clinical intervention or other attention is required.

Another objective is to provide a system and method for continuously monitoring an anastomosis.

Yet another objective is to provide a system and method for continuously monitoring a gastrointestinal anastomosis.

Still another objective is to provide a system and method for continuously monitoring the integrity of an organ inside a body.

A further objective is to provide a system and method for early and predictive detection of postsurgical complications particularly relevant to progression of the surgical site.

The above objectives are wholly or partially met by devices, systems, and methods according to the appended claims in accordance with the present disclosure. Features and aspects are set forth in the appended claims, in the following description, and in the annexed drawings in accordance with the present disclosure.

According to a first aspect, there is provided a system for monitoring a surgical site inside a body. The system includes an elongate sensing subsystem adapted so as to be placed in the body. The system further includes a communication subsystem generally situated outside of the body in communication with the elongate sensing subsystem. The elongate sensing subsystem further includes a sensor adapted to monitor physiological data from the surgical site.

The communication subsystem may be a network hub, a local network node, or the like, capable of establishing long range communication between the system and a data network. Alternatively or additionally, the communication subsystem may be a monitor, capable of communicating information to a user, patient, clinician and/or doctor about the state of the surgical site. The communication subsystem may also be a mobile computing device such as a mobile phone, an e-reader, a tablet, a media player, a mobile network node, or a pager. The communication subsystem may be a customized hub for establishing communication and transmitting physiologically relevant data between the elongate sensory subsystem and a data network such as an institutional data network, a LAN, a WAN, a cellular network, or the like.

The communication subsystem may include a sensor communication component that establishes and maintains communication with the elongate sensory subsystem. Alternatively, additionally, or in combination with the sensor communication component, the communication subsystem may further include a radio and antenna to communicate with the elongate sensory subsystem and/or communicate with an external network. The communication subsystem may also include a processor, memory, one or more clocks, a signal conditioning front end, a micropump, a controller, one or more valves, and/or one or more reservoirs. The communication subsystem may also include one or more secondary sensors including, without limitation, a temperature sensor, EKG sensor, EMG sensor, humidity sensor, altimeter, accelerometer, a piezoelectric sensor, a gyroscope, a pressure sensor, a pH sensor, a glucose sensor, or an acoustic sensor.

The physiological data communicated between the communication subsystem and the sensory subsystem may be raw data, metrics generated from the raw data pertaining to the progression or state of the surgical site, general patient information, calibration data, identification data, vital sign data or metrics formulated from the vital sign data, alerts, information about patient movement, information pertaining to trauma of the surgical site, or the like.

The elongate sensing subsystem may be a miniature device including a microcircuit, one or more sensors, and a substrate for interconnecting each of these elements. The elongate sensory subsystem may also include a power source, but it may be adapted to receive power from an external source such as an RF power source incorporated into the communication subsystem.

The elongate sensing subsystem may be generally shaped so as to be longer along one dimension than in other dimensions. In this way, the elongate sensing subsystem may be grain-like, worm-like, needle-like, or tape-like in shape, or similar. An elongate shape may be helpful in terms of maintaining subtle placement and retention near to a surgical site or within a hollow organ without causing excessive trauma or tissue damage. An elongate shape may be advantageous in terms of placement within a hollow tubular organ such as an artery, a vein, an intestine, an esophagus, or the like. In addition, an elongate shape may be advantageous for retention of a sensory subsystem in a hollow organ such as an intestine. Furthermore, such a shape may be advantageous for minimizing relative movement between the elongate sensory subsystem and either the surgical site or tissue relative to the surgical site. In this case, minimization of relative movement may lessen callous formation, and/or foreign body reaction, resulting in more consistent and reliable monitoring of the physiological data.

The elongate sensing subsystem may be an implantable medical device. It may be advantageous to implant the elongate sensing subsystem in close proximity to or at the surgical site. In a non-limiting example of monitoring of a gastrointestinal anastomosis implantation of the elongate sensing subsystem within the gastrointestinal system can be advantageous for ease of monitoring the anastomosis as well as care free removal of the device after the requisite monitoring period.

The elongate sensing subsystem may have an elastic modulus of less than 200 MPa, less than 75 MPa, less than 10 MPa, or less than 5 MPa. By elastic modulus it is meant the overall elastic modulus as measured during a tensile test along the elongate direction of the sensing subsystem. A sufficiently soft elongate sensing subsystem may be advantageous in terms of minimizing trauma caused to tissues in the vicinity of the sensing subsystem during use. In particular, it may be advantageous for the elongate sensing subsystem to be sufficiently soft such that the sensing subsystem can stretch along with local tissues in the vicinity of the surgical site during use.

The elongate sensing subsystem may be adapted to maintain communication with the communication subsystem while stretched at strains of up to 30%, up to 20%, up to 10%, or up to 5%. In several potential applications, the elongate sensing subsystem may be subjected to strains caused by patient movement, changes in posture, falls, externally applied pressures, or by contractions of local musculature. In the particular case of the gastrointestinal tract, motility related stresses may be substantial as the organ being monitored may actively try to remove the sensing subsystem. In such cases, it may be preferable for the elongate sensing subsystem to be able to stretch along with local tissues during use so as to minimize trauma to the surroundings tissues. Furthermore, such movements themselves may produce trauma at the surgical site. In order to maintain monitoring during these instances, it may be important for the elongate sensing subsystem to be adapted to maintain communication with the communication subsystem while under stretch.

The elongate sensing subsystem may include one or more bioadhesives adapted so as to anchor the elongate sensing subsystem in close proximity to or at the surgical site. Such bioadhesive attachment may be important for retaining the elongate sensing subsystem near or at the surgical site during monitoring.

Bioadhesives are biocompatible adhesives that can suitably bond the elongate sensing subsystem to tissues near a surgical site. Bioadhesives may be non-toxic, non-fouling, and biocompatible so as to help minimize the foreign body response during the monitoring process. Some suitable bioadhesives may include, without limitation, polysiloxanes, polyacrylates, polyisocyanate macromers or mixtures, fibrin sealants, albumin glue with gluteraldehyde as crosslinker, hydrogels such as those formed from chitosan and poly(ethylene glycol), gelatin-based adhesive with resorcinol-formaldehyde complex, oxidized polysaccharides with water-dispersible, multi-arm polyether amine, among others. The bioadhesives as noted above may also be sufficiently stable so as to retain the elongate sensing subsystem near to or at the surgical site during the postoperative recovery period but yet sufficiently biodegradable such that retention is only maintained for a reasonable period of time. In the case of a gastrointestinal anastomosis, the elongate sensory subsystem may be retained for up to 3 weeks, 2 weeks, or 1 week.

The bioadhesive may be located near the ends of the elongate sensing subsystem thus providing a means for bridging across the surgical site.

The elongate sensing subsystem may include one or more eyelets adapted so as to accept a suture or staple. Such sutures or staples may be biodegradable for easy detachment after a known period of retention. Some examples of suitable materials include without limitation, amino acid based families, polyester urethanes, polyester amides, polyester ureas, polythioesters, and polyesterurethanes.

The elongate sensing subsystem may be at least partially encapsulated with an elastomeric or gel coating selected from a group consisting of an anti-thrombogenic coating, a non-fouling coating and an anti-bioadhesive coating. Examples of suitable polymers may include without limitation, slow ion release polymers capable of releasing silver ions, carbon, platinum, silver sulfadiazine, chlorhexidine, cadexomer iodine, chlorhexidine gluconate, polyhexamethylene biguanide, antimicrobial peptides, and graphene. Such materials may be provided by Semprus Biosciences, Hydromer, Johnson and Johnson, Arrow International, Cook Critical Care, DSM Biomedical, and Edwards Lifesciences among others. Seprafilm™ (Genzyme Corporation) may additionally or alternatively be utilized for an anti-bioadhesive coating.

The elongate sensing subsystem may include an elastomeric capacitive strain and/or tension sensor. The elastomeric capacitive strain and/or tension sensor may further be arranged across the surgical site.

An elastomeric capacitive strain and/or tension sensor may include one or more thin dielectric membranes formed from an elastomer or gel; each sandwiched between thin elastic or structured electrodes. Due to changes in shape of the membranes under strain, a relationship between the impedance, particularly the capacitance, of the sensor and strain can be established. In a non-limiting example of an elastomeric membrane with high crosslink density, the creep properties of the membrane can be sufficiently low such that a relationship between the applied tension on the sensor and impedance of the sensor can be established.

The elongate sensing subsystem may include a flexible substrate onto which may be arranged a plurality of electrodes adapted to interface with the surgical site. One or more electrodes may be an insulated bioelectrode, which may possess longer term stability than un-insulated electrodes. An insulated bioelectrode may include a thin dielectric coating to isolate an underlying electrically conducting element. An insulated bioelectrode may be used to interact with surrounding tissues in an alternating current fashion with more effective interactions occurring at higher frequencies.

The elongate sensing subsystem may include a microcircuit in electrical communication with the electrodes arranged so as to monitor, measure, or detect one or more physiological or electrophysiological properties associated with the surgical site. Such physiological properties may include bioelectric activity, biopotentials, bioimpedance, bioimpedance tomography, motility, water content, pH, ionic strength, blood perfusion, and/or tissue oxygenation. A microcircuit in electrical communication with the electrodes may be configured to monitor, measure, or detect one or more disease states associated with the surgical site including ischemia, phagocytosis related to necrotic tissue consumption, inflammation, apoptosis, progression of wound healing, abscess formation, edema, a tear, and/or onset and/or development of a leak. Furthermore, a microcircuit in electrical communication with the electrodes may be configured to detect the presence of or measure concentrations of an analyte at the surgical site including hemoglobin, lactases, glycerin, water, oxygen, and/or matrix metalloproteinases.

The microcircuit may include a discrete microcircuit, a microcontroller, a microprocessor, a system-on-chip, application specific integrated circuit, a multi-chip mixed signal microcircuit, a field programmable gate array, a field programmable analog array, a digital signal processor, or the like. The microcircuit may include one or more components such as, without limitation, a sensor, a sensor front end, an analog to digital converter, a microprocessor, memory, a power source, power management hardware, flexible interconnects, and the like.

The elongate sensing subsystem may include an amperometric sensor. In another aspect, the elongate sensing subsystem may include one or more sensors including a potentiometer, a potentiostat, a bipotentiostat, a polypotentiostat, enzyme catalysis based sensors, a redox cell, a biofunctionallized ion-selective field effect transistor, and/or a potentiometric biosensor. The elongate sensing subsystem may include an impedance spectrometer.

The system and/or the elongate sensing subsystem may include one or more sensors selected from the group consisting of an accelerometer, a piezoelectric sensor, a gyroscope, a pressure sensor, an EKG sensor, an EMG sensor, a temperature sensor, a pH sensor, a glucose sensor, and/or an acoustic sensor.

The communication subsystem may be adhesively attached to the body, mounted on the body, attached to clothing on the body, or maybe a handheld device.

The communication subsystem may be a mobile computing device selected from the group consisting of a mobile phone, an e-reader, a tablet, a media player, a mobile network node, and a pager.

The elongate sensory subsystem and the communication subsystem may be in wireless communication.

The system may include one or more light sources arranged so as to emit light towards the surgical site and one or more photodetectors arranged so as to accept light from the surgical site. The light sources and photodetectors may be used to measure various physiological parameters of the tissues in and near to the surgical site including oxygen concentration, oxygenated hemoglobin concentration, deoxygenated hemoglobin concentration, matrix metalloproteases, and/or water content (oedema). The system may include a diffuse reflectance spectroscope, near infrared spectroscope, visible light spectroscope, surface plasmon resonant sensor, fluorescence spectroscope, ultra violet spectroscope, micro total analysis system, lab on a chip, dual- or multi-wavelength biosensor, miniaturized evanescent wave biosensor, pulsed oximeter, or the like in communication with the light source and photodetector to monitor the surgical site.

In another aspect, there is provided a system for monitoring a surgical site inside a body including a communication subsystem. The system further includes one or more elongate probes each with a distal end, a proximal end, and a length, the proximal end attached to the communication subsystem and the distal end adapted so as to be placed in close proximity to the surgical site. The system further includes a sensor in communication with the communication subsystem adapted to monitor physiological data from the surgical site.

The elongate probe may be shaped like a slender lace, tube, cord, or conduit connected to the communication subsystem. The elongate probe may be highly flexible so as to move with the tissues within the body so as to minimize tissue trauma during use.

The length of the elongate probe may be adjustable during use. In practical applications, estimating the distance between the surgical site and an adequate attachment point for the communication subsystem can be challenging. In the case of soft tissue surgery, such as during bowel surgery, the distance may be exceptionally challenging to determine, and may change significantly during the postoperative period. Adjusting the length of the elongate probe can be achieved by inclusion of a winding mechanism or spindle in the communication subsystem. Other methods for adjusting the elongate probe include cutting the probe, a sliding track, a folding mechanism, an external track such as could be adjusted by rotating the communication subsystem, etc. for adjusting the length of the elongate probe extending from the communication subsystem.

The sensor may include an elastomeric capacitive strain and/or tension sensor, an electrode based or bioelectrode based impedance sensor, or a sensor configured to monitor, measure or detect one or more physiological or electrophysiological properties associated with the surgical site. Such physiological properties may include bioelectric activity, biopotentials, bioimpedance, bioimpedance tomography, motility, water content, pH, ionic strength, blood perfusion, and/or tissue oxygenation. The sensor may also be configured to monitor, measure or detect one or more disease states associated with the surgical site including ischemia, phagocytosis related to necrotic tissue consumption, inflammation, apoptosis, progression of wound healing, abscess formation, edema, a tear, and/or onset and/or development of a leak. The sensor may further be configured to detect the presence of or measure concentrations of an analyte at the surgical site including hemoglobin, lactases, glycerin, water, oxygen, and/or matrix metalloproteinases. The sensor may include, without limitation, an amperometric sensor, a potentiometer, a potentiostat, a bipotentiostat, a polypotentiostat, enzyme catalysis based sensors, a redox cell, a biofunctionallized ion-selective field effect transistor, an impedance spectrometer, or a potentiometric biosensor. Furthermore, the sensor may include, without limitation, an accelerometer, a piezoelectric sensor, a gyroscope, a pressure sensor, an EKG sensor, an EMG sensor, a temperature sensor, a pH sensor, a glucose sensor, and/or an acoustic sensor. The sensory may also include, without limitation, a diffuse reflectance spectroscope, near infrared spectroscope, visible light spectroscope, surface plasmon resonant sensor, fluorescence spectroscope, ultra violet spectroscope, micro total analysis system, lab on a chip, dual or multi-wavelength biosensor, miniaturized evanescent wave biosensor, pulsed oximeter, or the like to monitor the surgical site.

The elongate probe may have an elastic modulus of less than 200 MPa, less than 75 MPa, less than 10 MPa, or less than 5 MPa. By elastic modulus is meant the overall elastic modulus as measured during a tensile test along the elongate direction of the elongate probe. Tissue trauma during the monitoring process may be reduced by making the elongate probe as soft and pliable as possible. The elongate probe may be formed from a silicone elastomer such as poly(dimethylsiloxane), viscoelastic gel, collagen, a porous core elastomer, a perfluoropolyether, a silicone-containing polyurethane, a sufficiently soft polyurethane, PFPE-PDMS block copolymers, polyisoprene, polybutadiene, fluoroolefin-based fluoroelastomers, an elastic protein such as resilin, and the like.

The elongate probe may includes a soft polymeric, elastomeric, or gel coating selected from the group consisting of an anti-thrombogenic coating, a non-fouling coating or an anti-bioadhesive coating. Non-limiting examples of suitable polymers may include slow ion release polymers capable of releasing silver ions, carbon, platinum, silver sulfadiazine, chlorhexidine, cadexomer iodine, chlorhexidine gluconate, polyhexamethylene biguanide, antimicrobial peptides, and graphene. Such materials may be provided by Semprus Biosciences, Hydromer, Johnson and Johnson, Arrow International, Cook Critical Care, DSM Biomedical and Edwards Lifesciences among others. Seprafilm™ (Genzyme Corporation) may additionally or alternatively be utilized for an anti-bioadhesive coating.

The sensor may be attached to the distal end of elongate probe. In this case, the sensor is generally miniaturized and adapted to interface with the surgical site or tissues in the vicinity of the surgical site. The sensor may be in communication with the communication subsystem via the elongate probe and/or via wireless communication protocols.

The sensor may be electrically and/or mechanically interfaced with the proximal end of the elongate probe. In this case, the sensor may interface with the surgical site via the elongate probe. Such configurations may be advantageous for systems including a reusable part such as the communication subsystem and a disposable part such as the elongate probe.

The system may include elastomeric optical fibers arranged so as to transmit light along the elongate probe between the communication subsystem and the surgical site. Elastomeric optical fibers allow for the transfer of light to or from the surgical site through the elongate probe. Elastomeric optical fibers may be formed from highly pure silicone elastomers. The elastomeric optical fiber may be embedded into the elongate probe, or may be provided separately running alongside the elongate probe. In addition, the elastomeric optical fiber may be arranged within a lumen of the elongate probe.

The system may include one or more soft polymeric or elastomeric conducting fibers arranged along the length of the elongate probe to provide electrical communication between the communication subsystem and the surgical site. One configuration may include a composite formed from a non-conducting elastomer such as a silicone or polyurethane and one or more electrically conducting filler materials such as metallic or carbon powders, or conjugated polymer fillers such as PEDOT doped with poly(styrenesulfonate) (PEDOT/PSS). Additionally or alternatively, a composite structure may be used to couple a thin film non-elastomeric conductor with an elastomeric substrate to form an equivalent to an elastomeric conducting fiber. In this case, the thin film non-elastomeric conductor is deposited on the surface of a stretched or preformed elastomeric substrate so as to form a series of buckled structures in operation. The buckled structures allow for the entire composite to stretch without damaging the non-elastomeric conductors. Stretchable electronic interconnections to connect rigid semiconducting islands may also be utilized. The electronic interconnections may be provided by meandering thin film traces and by lift off of traces from an elastomeric substrate such as silicone. Such an approach can also be used to create an equivalent to an elastomeric conducting fiber.

The system may include an electrically conducting microspring embedded longitudinally within the elongate probe to provide electrical communication between the communication subsystem and the surgical site. The electrically conducting microspring may include a helical coil microspring, a crimped microspring, etc. The microspring enables electrical communication between the communication subsystem and the distal end of the elongate probe and/or the surgical site. The microspring may be shaped so as to be sufficiently low modulus such that it does not adversely affect the overall elastic modulus of the elongate probe.

The system may include one or more light sources configured to emit light towards the surgical site, and one or more photodetectors for receiving light from the surgical site. The light sources and photodetectors may be used to measure various physiological parameters of the tissues in and near to the surgical site including oxygen concentration, oxygenated hemoglobin concentration, deoxygenated hemoglobin concentration, matrix metalloproteases, and/or water content (edema). The system may include a diffuse reflectance spectroscope, near infrared spectroscope, visible light spectroscope, surface plasmon resonant sensor, fluorescence spectroscope, ultra violet spectroscope, micro total analysis system, lab on a chip, dual or multi-wavelength biosensor, miniaturized evanescent wave biosensor, pulsed oximeter, or the like in operative communication with the light source and photodetector to monitor the surgical site.

The system may include a flexible substrate twisted into a helix arranged along the length of the elongate probe including electrical traces adapted to provide electrical communication between the communication subsystem and the distal end of the elongate probe.

The system may include one or more sensors arranged at a distal end of the elongate probe in electrical contact with the electrical traces.

The system may include a bioadhesive affixed to a distal end of the elongate probe adapted so as to fixedly retain the distal end of the elongate probe near to the surgical site. The bioadhesive may be biodegradable or bioabsorbable. Some examples of potentially suitable materials include amino acid based families, polyester urethanes, polyester amides, polyester ureas, polythioesters, and polyestcrurethanes.

The system or communication subsystem may include a micropump and one or more reservoirs, the reservoir in fluid communication with the micropump, the elongate probe further including a conduit arranged along the length of the elongate probe, the conduit adapted so as to provide fluid communication between the micropump, the reservoir, and/or the surgical site. The conduit may be arranged down the center line of the elongate probe, or it may be part of a multilumen elongate probe, whereby one or more lumens may provide bidirectional fluid communication between the reservoir, the micropump and/or the surgical site. In the case of a multilumen elongate probe, one or more lumens may provide fluid communication from the micropump and/or reservoir to the surgical site. One or more lumens of a multilumen elongate probe may provide fluid communication from the surgical site to the micropump and/or reservoir.

The micropump may include an electromagnetic micropump such as a rotary pump, peristaltic micropump, a syringe pump, or a solenoid driven pump, an active material micropump such as an electroactive polymer micropump, a shape memory material micropump, a piezoelectric micropump, or a piezoceramic micropump.

The micropump may be adapted to retrieve a fluid sample from the surgical site and deliver the fluid sample to the reservoir. The reservoir may include a reservoir integral to the system, a disposable attachable reservoir, a syringe, or the like. The reservoir may contain a medicament selected from the group consisting of antibiotics, anti-inflammatory agents, topical anaesthesia, antithrombogenic agents, and thromogenic agents. The micropump may be adapted to deliver the medicament from the reservoir to the surgical site. The micropump may configured to aspirate the surgical site. Aspiration may be valuable during withdrawal of the elongate probe after an adequate period of monitoring. Alternatively in the case of a heavily exuding surgical site, aspiration may be used to deliver exudates to the reservoir.

The communication subsystem may include an alerting component adapted to raise an alarm conditional on the physiological data. The alerting component may include an audible alarm, a visual alarm, a display, a vibratory alarm, a text messaging system, and a pager alert, or the like.

The system may include one or more sensors selected from the group consisting of an accelerometer, a piezoelectric sensor, a gyroscope, a pressure sensor, an EKG sensor, an EMG sensor, a temperature sensor, a pH sensor, a glucose sensor, and an acoustic sensor.

The communication subsystem and/or the elongate probe may include one or more sensors selected from the group consisting of an accelerometer, gyroscope, pressure sensor, EKG sensor, ECG sensor, EMG sensor, temperature sensor, pH sensor, glucose sensor, and an acoustic sensor.

The communication subsystem may be a mobile computing device such as a mobile phone, an e-reader, a tablet, a media player, a mobile network node, or a pager.

The communication subsystem and the elongate probe may be in wireless communication.

In some non-limiting examples, the surgical site may be an anastomosis performed on an organ selected from a group consisting of a colon, small intestine, bile duct, pancreas, stomach, esophagus, artery, vein, ureter, urinary bladder, urethra, and nerve. In particular, the surgical site may be a gastrointestinal anastomosis.

According to another aspect there is provided a system for monitoring a surgical site inside a body including an elongate probe in conjunction with an elongate sensory subsystem. The elongate sensory subsystem and the elongate probe are in operative communication with the communication subsystem as described herein. The combination of the elongate sensory subsystem and the elongate probe may be particularly advantageous for determining the state of a surgical site both inside and in close proximity to an organ.

According to yet another aspect there is provided a system for monitoring the integrity of an organ including a communication subsystem, an elongate probe with a distal end and a proximal end, the distal end adapted so as to be placed in close proximity to the organ, the proximal end attached to the communication subsystem, and an implantable sensory subsystem arranged for implantation inside the organ. The implantable sensory subsystem is in operable communication with the elongate probe and/or the communication subsystem thus forming a data stream. The communication subsystem includes an analytic component adapted to analyze the data stream.

According to another aspect is provided a method for determining the integrity of an organ including implanting a sensory subsystem inside the organ, placing an elongate probe in close proximity to the organ, communicating a data stream between the sensory subsystem and the elongate probe, and analyzing the data stream with an analytic component. The data stream may provide information pertaining to the integrity of the organ wall in the vicinity of sensory subsystem. In particular, the data stream may provide information pertaining to the development of thinning of the organ wall, development of an abscess in the organ wall, and/or the onset or development of a leak in the organ wall.

The analytic component may include, without limitation, a microcircuit, field programmable gate array, application specific integrated circuit, system-on-chip, microprocessor, digital signal processor, or the like. Additionally or alternatively the analytic component may be an offline resource such as a server, personal computer, or the like programmed to compute clinically relevant metrics from the incoming data stream. The analytic component may be incorporated into the communication subsystem or it may be in operable communication with the communication subsystem.

According to yet another aspect a method is provided for monitoring progression of a disease in an organ including implanting a sensory subsystem inside the organ, placing an elongate probe in close proximity to the organ, communicating a data stream between the sensory subsystem and the elongate probe, and analyzing the data stream with an analytic component.

According to another aspect is provided a method for monitoring the integrity of an anastomosis of an organ including, implanting a sensory subsystem inside the organ in close proximity to the anastomosis, placing an elongate probe in close proximity to the organ, communicating a data stream between the sensory subsystem and the elongate probe, and analyzing the data stream with an analytic component.

According to yet another aspect a system is provided for monitoring the integrity of an anastomosis of a hollow organ with a nervous system including, a communication subsystem, and a sensory subsystem in operable communication with the communication subsystem adapted to bridge the anastomosis. The sensory subsystem may include a sensor configured to monitor and/or compare electropotential signals from the nervous system on either side of the anastomosis. Electropotential signals in the vicinity of an anastomosis and particularly on either side of an anastomosis may become disrupted during the development of a complication such as formation of an abscess, thinning of the organ walls, and/or the onset or development of a leak.

The system may further include a sensor adapted to measure temperature of the anastomosis. Temperature changes in the vicinity of the anastomosis, particularly changes relative to the surrounding tissues, may be associated with onset of infection and/or development of tissue ischemia and/or an abscess.

The system may further include a sensor adapted to monitor bioimpedance across the anastomosis.

According to a further aspect a system is provided for monitoring the integrity of a gastrointestinal anastomosis with an enteric nervous system including, a sensory subsystem adapted to bridge across the gastrointestinal anastomosis. The sensory subsystem may include a sensor arranged to interface with the enteric nervous system in the vicinity of the gastrointestinal anastomosis and further adapted to monitor and/or compare electropotential signals from the enteric nervous system on either side of the anastomosis.

The system may further include a communication subsystem in communication with the sensory subsystem wherein, i.e. the communication subsystem is adapted to analyze the signals communicated from the sensor.

The system may further include a sensor adapted to measure a temperature of the gastrointestinal anastomosis. In particular, the system may also include a second temperature sensor adapted to measure a temperature of a segment of healthy tissue located near to the surgical site. Careful analysis of the temperature differential may be an early indicator of complications developing in the anastomosis.

According to a further aspect a system is provided for monitoring the integrity of an anastomosis including, a communication subsystem, and a sensory subsystem in communication with each other. The communication subsystem is adapted to interface with both sides of the anastomosis. The communication subsystem further includes a sensor configured to measure and/or compare blood perfusion on both sides of the anastomosis. A close comparison of perfusion on either side of the anastomosis may be an early indicator of the onset of ischemia. Additionally or alternatively, one or more sensors adapted to monitor and compare oxygen concentration on both sides of the anastomosis may help establish early onset of ischemia.

The system may further include a communication subsystem in communication with the sensory subsystem. The communication subsystem may be adapted to analyze the signals communicated from the sensor.

The system may further include a sensor adapted to measure a temperature of the gastrointestinal anastomosis. The system may also include a second temperature sensor configured to monitor the temperature of healthy tissue located near to the anastomosis. Careful analysis of the temperature differential may be an early indicator of complications developing in the anastomosis.

According to a further aspect a system is provided for monitoring the integrity or patency of an organ, transplant and/or a tissue/implant interface or vascular supply thereto after a surgical procedure, including an at least partially implantable sensory subsystem and a communication subsystem in communication with the sensory subsystem. The sensory subsystem further includes at least one sensor adapted to interface with the organ, transplant or tissue/implant interface or vascular supply thereto, a microcircuit, and at least one antenna. The sensory subsystem further includes an elongate housing arranged so as to interconnect each of these elements. The sensory subsystem may also include a power source such as, without limitation, a battery, fuel cell, energy harvesting devices, and the like.

The elongate sensory subsystem may be adapted to receive power from an external source such as an RF power source incorporated into the communication subsystem, or provided by ambient sources.

The sensor may be adapted to monitor oxygen perfusion, local anatomy changes, bioimpedance changes, or bioimpedance tomography changes within the organ, transplant, near to the tissue/implant interface, or within the vascular supply thereto.

The sensory subsystem may be generally shaped so as to be longer along one dimension than in other dimensions. Furthermore, the sensory subsystem may be adapted so as to have a flat or oblong cross section perpendicular to the long dimension. In this way, the elongate sensing subsystem may be grain-like, worm-like, needle-like, or tape-like in shape, or similar.

The sensory subsystem may further be flexible and/or stretchable. The combination of an elongate shape along with a flattened or oblong cross section in addition to being highly flexible and/or stretchable may be helpful in terms of maintaining subtle placement and retention near to a surgical site, along the outer surface of a vessel or organ, or within a hollow organ, without causing excessive trauma or tissue damage. An elongate shape may be of particular importance in terms of placement within a hollow tubular organ such as an artery, a vein, an intestine, an esophagus, or the like. In addition, an elongate shape may improve or enhance retention of a sensory subsystem in a hollow organ such as an intestine. Furthermore, such a shape may advantageously minimize relative movement between the elongate sensory subsystem and either the surgical site or tissue relative to the surgical site. In this case, minimization of relative movement may lessen callous formation, and/or foreign body reaction, resulting in more consistent and reliable monitoring of the physiological data.

The system may further include a withdrawal member adapted to connect the sensory subsystem with the outside of the body. The withdrawal member may be formed from, e.g. a cord, a braid, a fiber, or multifilament thereof. The withdrawal member may be formed from one or more polymers, metals, carbon fibers, or the like. In general, the withdrawal member may be coated with a biocompatible coating and/or a low friction or lubricious coating so as to minimize adhesion of the withdrawal member to the body during implantation.

The sensory subsystem may be adapted to monitor the tissue viability of the transplanted organ, tissue engineered construct, graft or tissue/implant interface. Additionally or alternatively, the sensory subsystem may be adapted to monitor the health of the vascular supply to a transplanted organ, tissue engineered construct, graft or tissue/implant interface prior to, during and/or after completion of a surgical procedure. The sensory subsystem may additionally or alternatively be adapted to monitor blood flow through the vascular supply prior to, during, and/or after completion of a surgical procedure. In one non-limiting example, the sensory subsystem may be adapted to monitor the patency of the right and/or left hepatic arteries in the vicinity of a liver.

The sensory subsystem may be sutured, stapled, glued or placed to the organ or tissue engineered construct prior to, during, or after completion of the surgical procedure. The sensory subsystem may further be embedded into an implant so as to monitor the tissue/implant interface after a surgical procedure.

Suitable organs may include, without limitation, a liver, colon, small intestine, bile duct, pancreas, stomach, esophagus, kidney, lung, artery, vein, ureter, urinary bladder, urethra, and nerve.

Tissue engineered constructs and vessels may be constructed and precursor materials selected from any suitable material. The tissue engineered construct may be fabricated from a range of cell sources including endothelial cells (EC), vascular smooth muscle cells (SMCs), fibroblasts, myofibroblasts, stem cells, and/or pericytes. In the case of a blood vessel construct including an adventitia, a contractile media, and an intima, the construct may be produced using cell-based tissue engineering methods. Vascular constructs can be produced from dermal fibroblasts, saphenous vein fibroblasts, and vascular SMCs. Relating to fabrication using tissue engineering methods, the tissue constructs may be fabricated in sheets to form adherent living tissue sheets. The sheets may then be rolled onto a tubular support and further cultured to form a tissue engineered vascular graft. A sensory subsystem may be incorporated into the tissue engineered construct at any stage in the fabrication process. In particular, a sensory subsystem may be incorporated between sheets during the rolling step of the fabrication process.

Additionally or alternatively, one or more sensory subsystems may be incorporated into a flexible mesh, biomaterial scaffold, or extracellular matrix (ECM) during or shortly after tissue engineered fabrication of a vascular graft.

The sensory subsystem may be directly attached to or embedded into a tissue engineered construct or vessel prior to implantation or even during the fabrication or growth process thereof. In the case of a vascular graft, the sensory subsystem may be inserted into or under the tunica externa of the graft, particularly between the tunica externa and the tunica media (smooth muscle). In the case of an arterial graft, the sensory subsystem may be inserted into tunica externa of the graft, placed between the serosa and either the tunica externa or the tunica media. Alternatively or additionally, the sensory subsystem may be attached to the serosa of an arterial graft, or to the tunica externa of a vascular graft. The sensory subsystem may be attached or embedded prior to implantation of the graft, during harvesting of the graft, or during fabrication of the graft, in the case of a tissue engineered graft.

According to a further aspect is provided, a sensory subsystem adapted for monitoring the patency of a stent. The sensory subsystem may be attached to the stent prior to, during or after placement of the stent. The sensory subsystem may be adapted to monitor flow of fluid through the stent during or after placement. Additionally, alternatively, or in combination, the sensory subsystem may be adapted to monitor tissue viability and/or anatomical changes in and around the stent during or after placement.

The stent may be selected from a group consisting of a vascular, ureteral, biliary, pancreatic, esophageal, bronchial, and tracheal stent.

According to a further aspect a method is provided for monitoring a surgical site within a body including: placement of a sensory subsystem prior to, during or after completion of a surgical procedure, monitoring the surgical site with the sensory subsystem for a period of seconds, minutes, hours, days, weeks or months, and removing the sensory subsystem from the body.

According to a further aspect a method is provided for monitoring a surgical site within a body including: placement of a sensory subsystem and attached withdrawal member prior to, during or after a surgical procedure, the sensory subsystem placed so as to interface with the surgical site and the withdrawal member placed so as to span from the sensory subsystem to the exterior of the body. The method may further include monitoring the surgical site with the sensory subsystem for a period of seconds, minutes, hours, days, weeks, or months, and removing the sensory subsystem from the body by pulling out the withdrawal member.

### BRIEF DESCRIPTION OF THE DRAWINGS

FIG. 1 shows an embodiment of an elongate sensing subsystem placed across an anastomosis formed in a large intestine in accordance with the present disclosure;

FIG. 2 shows an another embodiment of an elongate sensing subsystem with multiple electrodes arranged across an anastomosis in a large intestine in accordance with the present disclosure;

FIGS. 3a and 3b show a multi-view schematic of an embodiment of an elongate sensing subsystem including electrodes for interfacing with a surgical site in accordance with the present disclosure;

FIGS. 4a and 4b show a stretched and un-stretched view of an embodiment of an elongate sensing subsystem with an elastic strain or tension sensor in accordance with the present disclosure;

FIG. 5 shows an embodiment of an elongate sensing subsystem with bioadhesives patterned so as to bridge surgical site, in this case an anastomosis in accordance with the present disclosure;

FIG. 6 shows a schematic of an embodiment of an elongate sensing subsystem with multiple protective structures, arranged so as to protect as well as monitor a surgical site, in this case an anastomosis in accordance with the present disclosure;

FIG. 7 shows a connectivity diagram of an embodiment of a system for monitoring a surgical site in accordance with the present disclosure;

FIG. 8 shows another connectivity diagram of an embodiment of a system for monitoring a surgical site in accordance with the present disclosure;

FIG. 9 is a schematic view of another embodiment of a system including two elongate probes and a communication subsystem adapted for monitoring a surgical site, in this case a gastrointestinal anastomosis in accordance with the present disclosure;

FIG. 10 shows an embodiment for two elongate probes with bioadhesives at the distal ends for retaining the elongate probes in close vicinity to a surgical site, in this case a gastrointestinal anastomosis in accordance with the present disclosure;

FIG. 11 shows an another embodiment including two elongate probes with electrode sets arranged so as to monitor both sides of a gastrointestinal anastomosis in accordance with the present disclosure;

FIG. 12 shows an embodiment including an elongate probe placed externally to an organ and an elongate sensing subsystem attached internally to the organ, relatively near to a surgical site which in the example shown is a gastrointestinal anastomosis in accordance with the present disclosure;

FIG. 13 shows an another embodiment including an elongate probe with multiple electrodes draped along the outside wall of an intestine and an elongate sensing subsystem placed within the intestine near to an anastomosis in accordance with the present disclosure;

FIG. 14 shows an embodiment of a system for monitoring a surgical site including an elongate probe with multiple light sources and photodetectors along with an elongate sensing subsystem with a lightsource and photodetector placed inside of an intestine near to a surgical site, in this case an anastomosis in accordance with the present disclosure;

FIG. 15 shows multiple embodiments of elongate probes and elongate sensory subsystems arranged inside and near to an organ adapted for monitoring physiological data relevant to the state of the organ in accordance with the present disclosure;

FIG. 16 shows a schematic view of a communication subsystem with a mechanism for adjusting the length of an elongate probe in accordance with the present disclosure;

FIG. 17 shows a cross section of an embodiment of a distal end of an elongate probe with embedded elastomeric optical fibers arranged deliver light back and forth between a communication subsystem and a surgical site in accordance with the present disclosure;

FIG. 18 shows a cross section of an embodiment of a distal end of an elongate probe or the tip of an elongate sensing subsystem including embedded elastomeric optical fibers with a structured coating to act as lenses at the tips of the optical fibers in accordance with the present disclosure;

FIG. 19 shows a cross section of an embodiment of the distal end of an elongate probe or the tip of an elongate sensing subsystem with a light source and two photodetectors for interfacing with a surgical site in accordance with the present disclosure;

FIG. 20 shows a cross section of an another embodiment of the distal end of an elongate probe with helical electrical interconnects embedded along the elongate probe with a conduit for transferring fluids between the proximal and distal ends of the elongate probe in accordance with the present disclosure;

FIG. 21 shows multiple embodiments of the distal ends of elongate probes demonstrating an embedded spring-like conducting element for electrically communicating between the proximal and distal ends of an elongate probe and a conduit for providing fluid communication between the proximal and distal ends of an elongate probe in accordance with the present disclosure;

FIG. 22 is a schematic view of an embodiment of a system for monitoring a surgical site demonstrating an elongate sensing subsystem, an elongate probe and a communication subsystem with optional winding mechanism, micropump, medicament reservoir and sample reservoir in accordance with the present disclosure;

FIG. 23 shows a block diagram of a general layout for the electromechanical components of an embodiment of a communication subsystem in accordance with the present disclosure;

FIG. 24 shows a schematic of a fully implantable embodiment of a sensory subsystem for placement externally to an organ in accordance with the present disclosure;

FIG. 25 shows a schematic of an embodiment of a sensory subsystem attached to a withdrawal member in accordance with the present disclosure; and

FIG. 26 shows an arrangement of sensory subsystems arranged so as to monitor a liver transplant in accordance with the present disclosure.

### DETAILED DESCRIPTION

Particular embodiments of the present disclosure are described hereinbelow with reference to the accompanying drawings; however, the disclosed embodiments are merely examples of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. Like reference numerals may refer to similar or identical elements throughout the description of the figures. The term “clinician” refers to any medical professional (e.g., doctor, surgeon, nurse, or the like) performing a medical procedure involving the use of embodiments described herein.

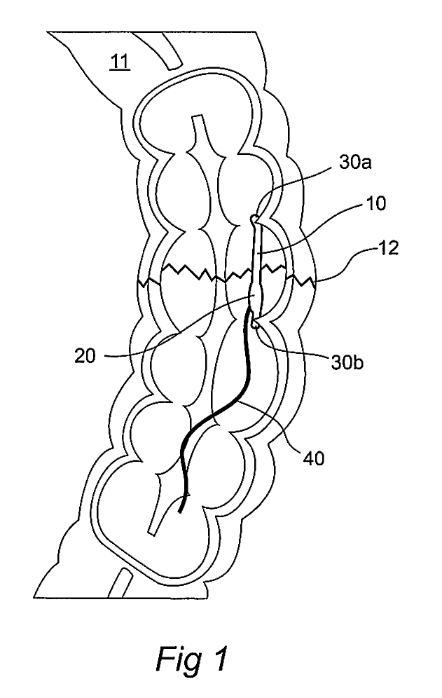

FIG. 1 shows an embodiment of an elongate sensing subsystem 10. In the example shown, the elongate sensory subsystem 10 is placed so as to bridge across a surgical site 12 (e.g. a gastrointestinal anastomosis) in an organ 11 (e.g. an intestine). The elongate sensing subsystem 10 is shown as placed within the organ 11. Additionally or alternatively, the elongate sensing subsystem 10 may be placed in close proximity to the surgical site 12 just outside the organ 11. The elongate sensing subsystem 10 includes two electrodes 30a30b arranged, in this example, near the ends of the elongate sensing subsystem 10. The electrodes 30a30b are generally used to interface with the tissue in the vicinity of or immediately upon the surgical site 12.

In the embodiment shown, the elongate sensing subsystem 10 also includes a microcircuit 20. The microcircuit 20 interfaces with the electrodes 30a30b during the monitoring process. In addition, the microcircuit 20 communicates with a remote communication subsystem (not explicitly shown). The microcircuit 20 generally monitors the surgical site 12 by electrical communication with the electrodes 30a30b. Electrical communication between the microcircuit 20 and the electrodes 30a30b can be provided by electrical traces embedded within the elongate sensing subsystem 10 (not explicitly shown). In addition, to establish communication between the elongate sensing subsystem 10 and a communication subsystem (not explicitly shown), the microcircuit 20 is equipped with a radio, associated electronics and an antenna 40. In this example, the antenna 40 is shown dangling along the axis of the organ 11. In an alternative example, the antenna 40 may be a chip antenna, trace antenna, or the like, embedded within the sensory subsystem 10. A dangling antenna 40 similar to what is shown, may be beneficial for use in applications requiring monitoring a surgical site 12 (e.g. an anastomosis) in a tubular organ 11 (e.g. an intestine, artery, vein, etc.). In such an application, a dangling antenna 40 helps to minimize the cross section of the sensing subsystem 10. A sensing subsystem 10 with minimal cross section may help to minimize disturbance to the local pathology and fluid flow through the tubular organ 11 thus improving retention, reducing tissue trauma and/or reducing fluid stasis around the sensing subsystem 10.

The microcircuit 20 may include discrete microelectronics, a microcontroller, a microprocessor, a system-on-chip, application specific integrated circuit, a multi-chip mixed signal microcircuit, a field programmable gate array, a field programmable analog array, a digital signal processor, or the like. The microcircuit 20 may include one or more sensors, a sensor front end, an analog to digital converter, a microprocessor, memory, a power source, power management hardware, flexible interconnects and the like.

The elongate sensory subsystem 10 may include a sensor (not explicitly shown) that would generally be embedded within the sensory subsystem 10 and/or included as part of the microcircuit 20.

Sensors included within the elongate sensing subsystem 10 and/or the microcircuit 20 may include, without limitation, an amperometric sensor, a potentiometer, a potentiostat, a bipotentiostat, a polypotentiostat, enzyme catalysis based sensors, a redox cell, a biofunctionallized ion-selective field effect transistor, an impedance spectrometer, or a potentiometric biosensor. Furthermore, the sensor may include, without limitation, an accelerometer, a piezoelectric sensor, a gyroscope, a pressure sensor, an EKG sensor, an EMG sensor, a temperature sensor, a pH sensor, a glucose sensor and/or an acoustic sensor. The sensor may additionally or alternatively include, without limitation, a diffuse reflectance spectroscope, near infrared spectroscope, visible light spectroscope, surface plasmon resonant sensor, fluorescence spectroscope, ultra violet spectroscope, micro total analysis system, lab on a chip, dual or multi-wavelength biosensor, miniaturized evanescent wave biosensor, pulsed oximeter, or the like to monitor the surgical site 12.

The electrodes 30a30b are generally arranged so as to interface with the tissues around a surgical site 12, the fluids in the vicinity of the surgical site 12, or the tissues within the surgical site 12. The electrodes 30a30b at least partially formed from an electrically conducting material such as, without limitation, platinum, silver, silver/silver chloride, carbon materials (e.g. activated carbon, graphite, graphene, etc.), gold, intermetallics, stainless steel, and the like. The electrodes 30a-30b may further include a radiopaque (e.g. radiodense) material such as, without limitation, platinum, gold, zirconium, palladium-based alloys, tungsten, tantalum, and the like. Such radiopaque materials may be advantageous for imaging placement of electrodes 1125-1129 in vivo. Additionally or alternatively, the electrodes 30a30b may include titanium nitride or cermets as a thin protective biocompatible layer. In general the electrodes 30a30b may be formed from a printable ink including a binder and conducting filler. The electrodes 30a30b may further include a conjugated polymer such as PEDOT, polyanaline, polypyrrole, polythiophenes, polyacetylene and the like. The electrodes 30a30b may include a radio opaque component so as to improve visualization with various imaging methods. The electrodes 30a30b may also be insulated bioelectrodes generally including a conducting electrode material with a thin dielectric overcoat. The dielectric overcoat may improve long term stability of the electrodes when placed in contact with live tissues.

The elongate sensing system 10 may be soft and elastic so as to minimize tissue trauma during placement and use. In this sense, the components of the elongate sensing system 10 may be sufficiently soft and elastic so as to move with the surrounding tissues. In the case that an embedded microcircuit 20 is electrically interfaced with electrodes 30a30b or another sensor arranged elsewhere on the sensory subsystem 10, interconnects may be sufficiently elastic such that the entire structure is rendered elastic. In this example, interconnects may be formed from elastic and/or elastomeric conducting elements, conducting polymers, or structured conducting elements forming a tortuous path on an elastomeric substrate embedded within the elongate sensory subsystem 10. Depending on the manner in which the interconnects may be structured, such interconnects may be repeatedly stretched without appreciable damage accumulation up to 10% stretch, 30% stretch, 50% stretch, or up to 100% stretch. In one non-limiting example, the interconnects may be formed through application of a microtextured or micropatterned mold followed by printing or vacuum deposition of a thin conducting layer onto the microtextured surface. Additionally or alternatively, stretchable interconnects can be achieved through careful application of electrically conducting yams, springs, or similar structures that form tortuous pathways for stiffer materials to flex during extension.

The elongate sensing system 10 may also include a power source, shown in FIG. 1 as integrated into the microcircuit 20. Additionally or alternatively, the elongate sensing system 10 may include one or more power sources attached along the length of the elongate sensing system 10, provided in electrical communication with the microcircuit 20. In general, a suitable power source may be a primary (e.g., non-rechargeable) microbattery or a secondary (e.g., rechargeable) microbattery. Additionally or alternatively, the sensing subsystem 10 may obtain power from an external source. In this case, power may be provided wirelessly by an external RF power source. To facilitate accepting power from an external source, the elongate sensing subsystem 10 may include an antenna 40, for coupling with the RF power source as well as have power management hardware to provide usable power from the harvested signals. Additionally or alternatively, the sensing subsystem 10 may be powered via conduction through the body by an externally applied source. The sensing subsystem 10 may additionally or alternatively be powered by means of an energy harvesting device, which may harvest ambient RF energy, thermal energy, mechanical energy from the surroundings. In addition, the elongate sensory subsystem 10 may be powered by a micro fuel cell, a radiation based battery technology, or the like.

In some applications, the sensing subsystem 10 may be physically attached to tissues in the vicinity of the surgical site 12. The sensing subsystem 10 may be attached using any suitable manner of attachment, such as, without limitation, mechanical fasteners, hooks or barbs, sutures, adhesives, or the like. Hooks and barbs can be an advantageous way to interface with the surgical site 12 in that they can be constructed to conduct electricity as well as can be made sufficiently biodegradable that the fixation points will weaken and break away from the body after a sufficiently long period of time. In the case of attachment by a suture, the suture may be formed from cotton, silk, nylon and/or polypropylene among other common materials. The suture materials may be biodegradable including such materials as surgical gut, chromic suture materials (generally type A or Type B), polyglycolic acid, polylactic acid, polydioxanone, and caprolactone amongst others.

The attachment means may also include a bioadhesive. In this case, depending on the location and function, the bioadhesive may be a non-conducting, microporous and/or ion conducting. An ion conducting bioadhesive may facilitate easily implemented yet reliable electrode-body interactions during the monitoring process.

Bioadhesives may be non-toxic, non-fouling and biocompatible so as to help minimize the foreign body response during the monitoring process. Some suitable bioadhesives may include, without limitation, polysiloxanes, polyacrylates, polyisocyanate macromers or mixtures, fibrin sealants, hydrogels such as those formed from chitosan and poly(ethylene glycol), gelatin based adhesive with resorcinol-formaldehyde complex, oxidized polysaccharides with water-dispersible, multi-arm polyether amine, protein based adhesives such as those formed from collagen, albumin or gelatin, in particular albumin glue with gluteraldehyde as a crosslinker, among others. The bioadhesives as noted above may also be sufficiently stable so as to retain the elongate sensing subsystem near to the surgical site during the postoperative recovery period but yet sufficiently biodegradable such that retention is only maintained for a reasonable period of time. In the case of a gastrointestinal anastomosis, the elongate sensory subsystem may be retained for up to 3 weeks, 2 weeks, or 1 week.

Bioadhesives may also be electrically or ionically conductive such as provided by ionically conductive hydrogels including dispersions of water soluble salts, silver chloride, and the like. The bioadhesive may also be a part of a high impedance electrode, capable of obtaining high frequency interactions with adjacent tissues while forgoing DC interactions. A non-limiting example of a high impedance electrode material is EXH 585 adhesive available from FLEXcon Company Inc. of Spencer, Mass. Other non-limiting examples include conductive carbon coatings, intrinsically conducting polymers such as Poly(3,4-ethylenedioxythiophene) (PEDOT) available commercially as Clevios™ from Heraeus, carbon nanotube dispersions such as Super HiPCO™ from Carbon Nanotechnologies Inc. of Houston, Tex. In addition, the bioadhesives may be provided in the form of an ion-conducting membrane so as to provide fluid exchange across the bioadhesive in addition to adhesive capabilities in a single structure.

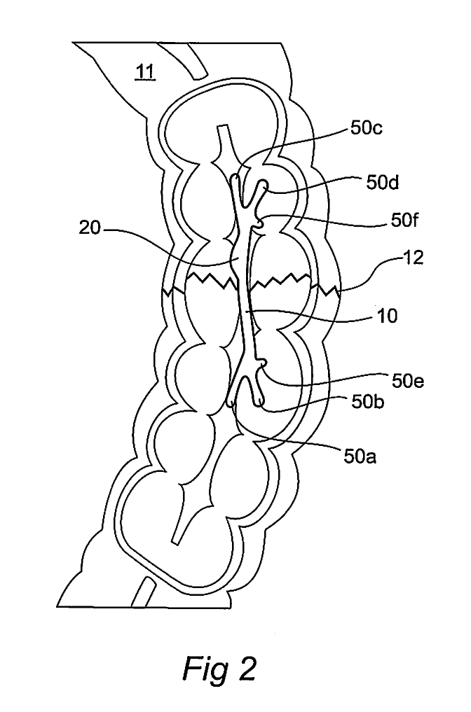

FIG. 2 shows another embodiment of an elongate sensing subsystem 40 having multiple electrodes 50a-f arranged across a surgical site 12 (e.g. an anastomosis) in a hollow organ 11 (e.g. a large intestine). In addition to all of the variations highlighted in the previous figure, this embodiment of the elongate sensing subsystem 40 includes multiple electrodes 50a-f and a microcircuit 45 arranged so as to interface with the electrodes 50a-f. Although 6 electrodes 50a-f are shown in the figure, more or fewer electrodes could be provided to achieve either similar or alternative functionality depending on the surgical site. Optionally, extra electrodes may be used to enhance impedance tomography measurements in the vicinity of the surgical site 12, while fewer electrodes may be sufficient for monitoring biosignals and/or bioimpedance across the surgical site 12.

The electrodes 50a-f are arranged so as to interface between those electrodes 50a, b, e arranged to one side of the surgical site 12 (e.g. an anastomosis) and those electrodes 50c, d, f arranged on the opposing side of the surgical site 12 (e.g. an anastomosis). Electrically interfaced sensing across a surgical site 12 in general, or in the case shown where the surgical site 12 is an anastomosis, may provide detailed information regarding the progression of the healing process. In particular, monitoring biosignals may be advantageous in regards to tracking the healing process of the surgical site 12. In the case of an organ with a highly active nervous system, monitoring signals produced by the nervous system and in particular interactions between neurons on either side of the surgical site 12 may be of particular advantage. In the case of a gastrointestinal anastomosis surgical site 12 in an organ 11 (e.g. colon), the biosignals produced in the enteric nervous system may provide information regarding the progression of ischemia, abscess formation, breakdown of neuron interactions across the surgical site and/or gap progression in the wall of the organ 11, which could be early indicators of the formation of a leak. The electrodes 50a-f may further be arranged so as to stimulate the adjacent enteric nervous system and monitor the response there from. Such responses may be advantageous in assessing the state of the surgical site 12 and surrounding tissues.

The electrodes 50a-f, may be configured such that a group of electrodes 50a b, e or 50c, d, f may measure biosignals, bioimpedance, impedance tomography, or the like to one side of the surgical site 12. In this case, the electrodes 50a, b, e or 50c, d, f may form an electrochemical cell including an active electrode 50a or 50c, a counter electrode 50b or 50d, and a reference electrode 50e or 50f. In this configuration, the electrochemical cell may sense the properties of tissue on a single side of the surgical site 12. This information may be used to assess the health of the local tissues, but may also be used to provide reference information for use in comparison with measurements pertaining to the tissues in the surgical site 12. The electrodes 50a-f, may also communicate in an AC fashion to measure bioimpedance spectra on either side of the surgical site 12 as well as across the surgical site 12. Such adjustable electrode interconnections allow for more information to be obtained about both the surgical site 12 as well as the surrounding tissues.

In addition to the previous descriptions of microcircuit 20, microcircuit 45 for this embodiment may be arranged to flexibly interface with the electrodes 50a-f so as to make the aforementioned measurements. The microcircuit 45 may also include hardware or be programmed so as to analyze and compare the above signals in order to make predictions about the progression of healing of the surgical site.

As seen in the FIG. 1, additional sensors in the elongate sensing subsystem 40 may be beneficial for further assessing the surgical site 12. In particular, a large stretch, soft strain gauge as well as one or more temperature sensors may be advantageous in such assessments.

The microcircuit 45 may be adapted to compile data from multiple independent sensors and electrodes 50a-f and to determine clinically relevant metrics regarding the state of the surgical site 12 from such multifaceted data. Furthermore, the microcircuit 45 may monitor data over time to readily predict changes in the state of the surgical site 12.

The electrodes 50a-f, as well as the electrodes 30a, b of the previous embodiments, may include biomolecules or biomarkers for detecting biological species in and around the surgical site. Such information may be advantageous for determining the onset of infection or necrosis in and/or around the surgical site and may prove advantageous in predicting the onset of postoperative complications.

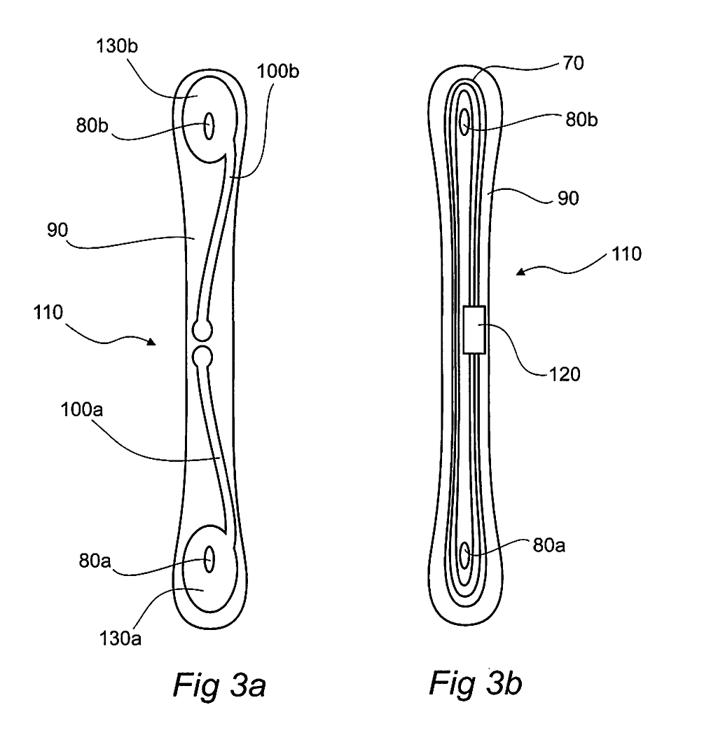

FIGS. 3a and 3b show a multi-view schematic of an embodiment of an elongate sensing subsystem 110 including electrodes 130a, b for interfacing with a surgical site (not explicitly shown). Although the elongate sensing subsystem 110 may include some or all of the features discussed in previous embodiments, it may include the features discussed herein with respect to the present embodiment. The elongate sensory subsystem 110 includes a substrate 90. The substrate 90 may be polymeric or elastomeric so as to provide a soft, flexible sensory subsystem 110 for use near soft tissues without undue mechanical trauma. The substrate 90 may provide a foundation for placement of electrical traces 100a, b, electrodes 130a, b, a microcircuit 120, an embedded antenna 70 and (optional) eyelets 80a, b. The electrodes 130a, b are generally arranged so as to electrically interface with adjacent fluids or tissues for impedance or biosignal based assessment of a surgical site. The electrodes 130a, b may include similar features to those described previously. Furthermore, although only two electrodes 130a, b are shown in the FIG. 3a or 3b, there may be more electrodes without altering the spirit or scope of the disclosed sensory subsystem 110. The optional eyelets 80a, b may be arranged at or within the electrodes 130a, b so as to anchor the electrodes 130a, b to adjacent tissues. The eyelets 80a, b may be advantageous for interfacing a suture with the elongate sensing subsystem 110. The eyelets 80a, b may be located away from the electrodes 130a, b for attachment of alternative segments of the elongate sensing subsystem 110 to adjacent tissues. The functionality of the eyelets 80a, b may additionally or alternatively be provided with bioadhesives or similar attachment means. The electrodes 130a, b may be fashioned so as to enhance the mechanical strength of the elongate sensing subsystem 110, which may prevent a suture from tearing an eyelet 80a, b during use.

The electrodes 130a, b are electrically interfaced with the microcircuit 120 by means of the traces 100a, b. The traces 100a, b may be formed from elastomeric electrically conducting materials, microformed or microtextured electrically conducting materials, or the like. The traces 100a, b may also form a large stretch capacitive strain sensor. It may be advantageous to include a capacitive strain sensor into an elongate sensory subsystem 110 with more electrodes than those electrodes 130a, b shown in the figure.

In this embodiment, the microcontroller 120 is also electrically interfaced with an integrated antenna 70 arranged on the substrate 90. The integrated antenna 70 may be used to communicate with a remote communication subsystem (not explicitly shown), an elongate probe (not explicitly shown), or an adjacent medical device (not explicitly shown). The antenna 70, especially in conjunction with the traces 100a, b and the electrodes 130a, b may effectively form a soft elastic strain and/or tension gauge.

The layout of the antenna 70 may be configured to minimize impedance variations with stretch of the elongate sensing subsystem 110. In this configuration, the elongate sensory subsystem 110 may maintain wireless communication with an external communication subsystem (not explicitly shown) while stretched during use. Such capability may vastly improve the reliability of monitoring the surgical site in practice.

The antenna 70 may be used so as to measure bioimpedance or monitor biosignals in and around the surgical site (not explicitly shown) in addition to facilitating communication with a communication subsystem (not explicitly shown). In this configuration, the microcircuit 120 may include hardware for driving and for sensing signals from the antenna 70 at a frequency or over a frequency range suitable for monitoring biosignals or bioimpedance of the surrounding tissues, while also being able to drive and to sense signals from the antenna 70 at or over a frequency range suitable for wireless communication with an external communication subsystem.

The antenna 70 may also provide a means for transmitting and/or receiving data and/or test signals with an elongate probe, optionally placed near to the organ or elongate sensing subsystem 110. In such an embodiment, the microcircuit 120 may send a signal via the antenna 70 that is received and analyzed by an elongate probe (not explicitly shown) or communication subsystem. Additionally or alternatively, the antenna 70 may receive a signal from a communication subsystem or elongate probe. The received signal may be analyzed by the microcircuit 120. The microcircuit 120 may analyze the signal for trends in terms of the phase content, frequency content, amplitude, spectrum, or similar aspects of the signal, any or all of which may be indicative of progression of healing in the surgical site.

The antenna 70 may be constructed from a range of elastic composite electrically conducting materials. Some elastomeric electrically conducting materials have been previously discussed. Suitable materials may include elastic and/or elastomeric conducting elements, conducting polymers, or structured conducting elements forming a tortuous path on the surface of the elastomeric substrate 90 embedded within the elongate sensory subsystem 110. Depending on the manner in which the conductors may be structured, such conductors may be repeatedly stretched without appreciable damage up to 10% stretch, 30% stretch, 50% stretch, or up to 100% stretch. In one example, the conductors may be formed though application of a microtextured or micropatterned mold followed by printing or vacuum deposition of a thin conducting layer onto the microtextured surface. Additionally or alternatively, stretchable conductors can be achieved through careful application of electrically conducting yarns, springs or similar structures that form tortuous pathways for stiffer materials to behave elastically during extension.

The elongate sensing subsystem 110 may be at least partially encapsulated with an elastomeric or gel coating selected from a group consisting of an anti-thrombogenic coating, a non-fouling coating and an anti-bioadhesive coating. Non-limiting examples of suitable polymers may include slow ion release polymers capable of releasing silver ions, carbon, platinum, silver sulfadiazine, chlorhexidine, cadexomer iodine, chlorhexidine gluconate, polyhexamethylene biguanide, antimicrobial peptides, and graphene. Seprafilm™ (Genzyme Corporation) may additionally or alternatively be utilized for an anti-bioadhesive coating.

As outlined in the previous figures, additional sensors in the elongate sensing subsystem 110 may be beneficial for further assessing the surgical site. In particular, a large stretch, soft strain/tension sensor as well as one or more temperature sensors may be advantageous in assisting with the assessment.

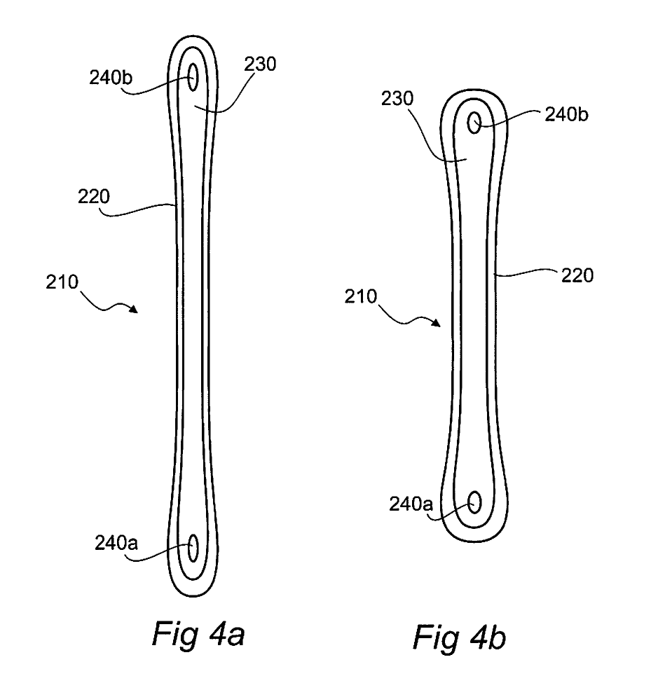

FIGS. 4a and 4b show a stretched and un-stretched view of an embodiment of an elongate sensing subsystem 210 including an elastic strain and/or tension sensor. In one embodiment, the elastic strain and/or tension sensor may be formed from one or more elastomeric substrates 220 sandwiched between two or more electrodes 230. Only one electrode 230 is shown. The elastic strain and/or tension sensor may further include an attachment means located near the ends of the substrate 220. In the embodiment shown, the attachment means are formed from eyelets 240a, b. The attachment means may also be formed from adhesives, hook and loop structures, barbs, or the like. In general, strain and/or tension measurements can be ascertained from changes in the electrical impedance and, in particular, the capacitance of the sensor when stretched.

The elastic strain and/or tension sensor may be formed from multiple layers of the above substrate 220 and electrodes 230. In this case, the capacitance of the sensor may be appreciably increased without substantially increasing the size of the sensor. In another embodiment, the elastic strain and/or tension sensor may be formed by rolling a substrate 220 and electrodes 230 into a tight elastic tube.

The electrodes 230 may be formed from elastic and/or elastomeric conducting elements, conducting polymers, or structured conducting elements forming a tortuous path on the elastomeric substrate 220 embedded within the elongate sensory subsystem 210. In an embodiment including elastomeric composite electrodes 230, the electrodes 230 may be formed from an elastomeric conducting composite generally including electrically conducting micro- or nano-sized particulates dispersed into an elastomeric matrix. The particulates may be formed from metals, graphite, carbon black, graphene, inherently conducting polymers, or the like.

Depending on the manner in which the electrodes 230 may be structured, such electrodes 230 may be repeatedly stretched without appreciable damage accumulation up to 10% stretch, 30% stretch, 50% stretch, or up to 100% stretch. In one non-limiting example, the electrodes 230 may be formed though application of a microtextured or micropatterned mold followed by printing or vacuum deposition of a thin conducting layer onto the microtextured surface. Additionally or alternatively, stretchable electrodes 230 can be achieved through careful application of electrically conducting yarns, springs or similar structures that form tortuous pathways for stiffer materials to flex during extension.

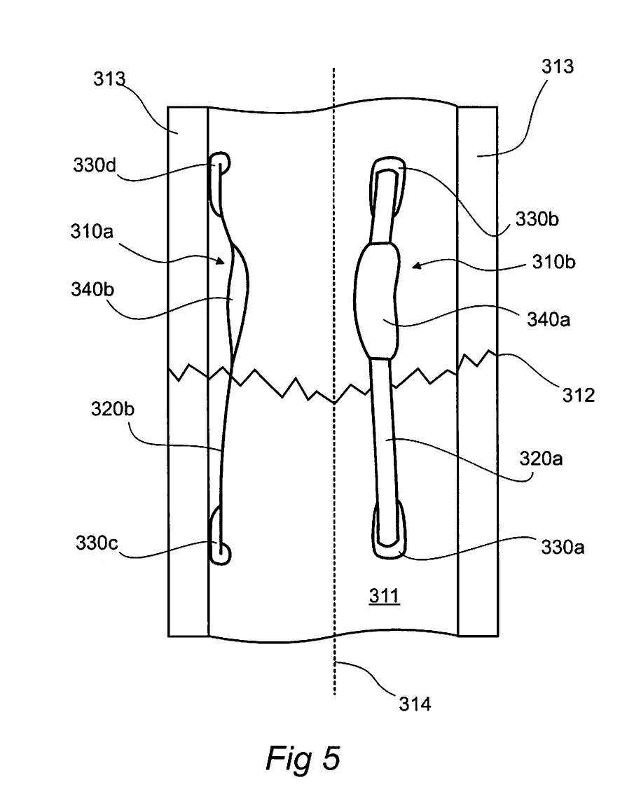

FIG. 5 shows two embodiments of an elongate sensing subsystem 310a, b arranged so as to bridge a surgical site 312 (e.g. a hollow organ, an anastomosis, a gastrointestinal anastomosis, a vascular anastomosis, a renal anastomosis, etc.). As shown in FIG. 5 the organ 311 is hollow and has a lumen axis 314 arranged internally and along the length of the organ 311. In general the elongate sensing subsystem 310a, b is oriented in the same direction as the lumen axis 314 The plurality of elongate sensing subsystems 310a, b are shown to demonstrate the possibility for coordinated sensing from multiple sensing subsystems 310a, b as well as the ability to place multiple sensing subsystems 310a, b at a surgical site 312. Multiple sensing subsystems 310a, b may be advantageous for improving the reliability of assessing the surgical site 312. In addition the inclusion of multiple sensing subsystems 310a, b may be advantageous for attaining more local information about a surgical site 312, such as where a leak or abscess is developing.

The elongate sensing subsystem 310a, b includes a flexible and/or elastomeric substrate 320a, b which generally provides shape and structure. The sensing subsystem 310a, b further includes one or more bioadhesive elements 330a, b, c, d attached to the substrate 320a, b. Here the adhesive elements 330a, b, c, d are shown arranged near the ends of the substrate 320a, b. The bioadhesives are provided so as to interface with the lumen wall 313 on either side of the surgical site 312. Some properties and examples of suitable bioadhesives were discussed previously herein.

Particularly interesting for the embodiment shown, the bioadhesive regions 330a, b, c, d may be electrically or ionically conductive such as provided by ionically conductive hydrogels including dispersions of water soluble salts, silver chloride, and the like. The bioadhesive elements 330a, b, c, d may also be a part of a high impedance electrode capable of obtaining high frequency interactions with adjacent tissues while forgoing DC interactions. A non-limiting example of a high impedance electrode material is EXH 585 adhesive available from FLEXcon Company Inc. of Spencer, Mass. Other non-limiting examples include conductive carbon coatings, intrinsically conducting polymers such as Poly(3,4-ethylenedioxythiophene) (PEDOT) available commercially as Clevios™, carbon nanotube dispersions such as Super HiPCO™ from Carbon Nanotechnologies Inc. of Houston, Tex. In addition, the bioadhesive regions 330a, b, c, d may be provided in the form of an ion-conducting membrane so as to provide fluid exchange across the bioadhesive region 330a, b in addition to adhesive capabilities in a single structure.

The elongate sensing subsystem 310a, b further includes a soft encapsulated region 340a, b. The soft encapsulated region 340a, b may be partially or fully coated over the substrate 320a, b depending on the application needs. In general the encapsulated region 340a, b is used to electrically and/or mechanically isolate microcircuits, traces, optical sensors as well as other sensitive or intrusive elements that may need to be separated from the surrounding tissues during use. The encapsulated region 340a, b may be formed from an elastomer, such as, without limitation, a silicone, polyisoprene or soft polyurethane. Additionally or alternatively, the encapsulated region 340a, b may be formed from a range of soft polymeric, elastomeric, or gel coatings selected from the group consisting of an anti-thrombogenic coating, a non-fouling coating, and an anti-bioadhesive coating.

Non-limiting examples of suitable polymers may include slow ion release polymers capable of releasing silver ions, carbon, platinum, silver sulfadiazine, chlorhexidine, cadexomer iodine, chlorhexidine gluconate, polyhexamethylene biguanide, antimicrobial peptides, and graphene. Such materials may be provided by Semprus Biosciences, Hydromer, Johnson and Johnson, Arrow International, Cook Critical Care, DSM Biomedical and Edwards Lifesciences among others. Seprafilm™ (Genzyme Corporation) may additionally or alternatively be utilized for an anti-bioadhesive coating.

The encapsulated region 340a, b may also provide release of a therapeutic agent such as a wound cleanser, antiseptic, hydrogen peroxide, povidone iodine, chlorhexidine diacetate, sodium hypochlorite, antibiotics, bacitracin, polymyxin B, neomycin, silver sulfadiazine, nitrofurazone ointment, gentamicin sulfate, cefazolin, granulation tissue suppressing agents, corticosteroids, activated macrophage supernatant, aloe vera, comfrey (symphytum officinale), eucalyptus, hydrastis canadensis, melaleuca, alternifolia, thymol, trypsin, elase, Granulex™, honey, Lanolin, phenytoin, galium nitrate, gentian violet, recombinant vasoactive protein, scarlet oil, aminoplex,sugardine, tripeptide-copper complex, vitamin E, and the like. The release of a therapeutic agent may be passive or may be driven or activated by a mechanism such as an electrophoretic element, iontophoretic element or similar at least partially located within the encapsulated region 340a, b. Other electrodes necessary for active release of a therapeutic agent may be provided on an exposed region of the substrate 320a, b or somewhere near or on the bioadhesive region 330a, b.

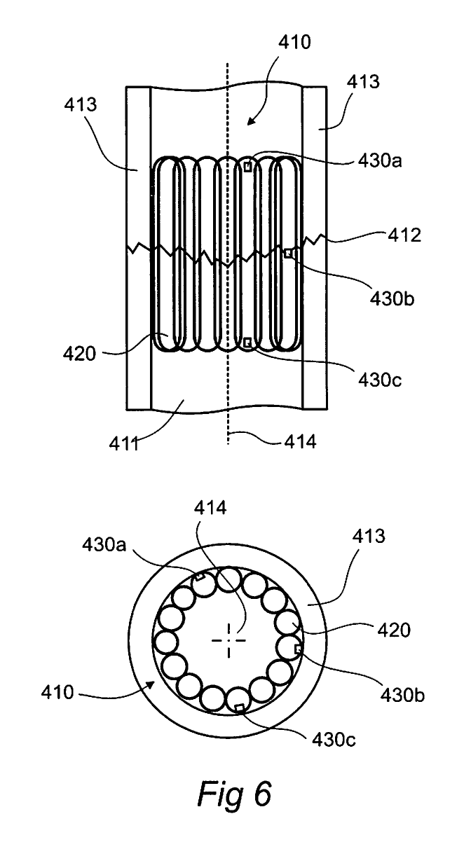

FIG. 6 shows a schematic of an embodiment of an elongate sensing subsystem 410 with multiple protective structures, arranged so as to protect and/or monitor a surgical site 412 (e.g. an anastomosis, a gastrointestinal anastomosis, a vascular anastomosis, a renal anastomosis, etc.). The elongate sensing subsystem 410 includes one or more elongate structures 420 adapted so as to be arranged within an organ 411 (e.g. a hollow organ, an intestine, a vein, an artery, etc.) in contact with the lumen wall 413, generally coaxial to the lumen axis 414.

The elongate structure 420 may include a microcircuit, a power source, an antenna, and/or similar components, in order to both monitor the surgical site 412 and communicate relevant data with a communication subsystem. The elongate structure 420 may also include a sensor 430a, b, c for collecting physiological data from the surgical site 412. The elongate structure 420 may generally be elastomeric and flexible so as to minimize trauma on the surrounding tissues. The elongate structure 420 may also be sufficiently low profile so as to minimally affect fluid flow within the organ being monitored. This can be particularly important for monitoring cardiovascular organs, where disturbance of blood flow can lead to clot formation, or during monitoring of gastrointestinal organs where disturbance of the flow can lead to dead zones where foreign matter can collect, potentially leading to complications.

The elongate structure 420 may be formed from a range of elastomeric materials. Additionally or alternatively the elongate structure 420 may be formed from a group consisting of an anti-thrombogenic coating, a non-fouling coating or an anti-bioadhesive coating. Non-limiting examples of suitable polymers may include slow ion release polymers capable of releasing silver ions, carbon, platinum, silver sulfadiazine, chlorhexidine, cadexomer iodine, chlorhexidine gluconate, polyhexamethylene biguanide, antimicrobial peptides, and graphene. Such materials may be provided by Semprus Biosciences, Hydromer, Johnson and Johnson, Arrow International, Cook Critical Care, DSM Biomedical and Edwards Lifesciences among others. Seprafilm™ (Genzyme Corporation) may additionally or alternatively be utilized for an anti-bioadhesive coating.

The sensors 430a, b, c may be any of the previously discussed sensors. In particular the sensors 430a, b, c may be adapted to monitor activity of the enteric nervous system, perfusion of tissues, bioimpedance, strain, temperature profiles, or similar physiological function at and/or near the surgical site.

In one particular embodiment relevant to all previous figures as well as to that of FIG. 6, the elongate sensing subsystem 10, 40, 110, 210, 310a,b, 410, may include an array of temperature sensors arranged so as to bridge the surgical site 12, 312, 412. In this embodiment, the temperature sensors may provide a detailed map of temperature variation in and around the surgical site 12, 312, 412. Such information may be advantageous for early detection of ischemia and/or the onset of infection of the surgical site 12, 312, 412.

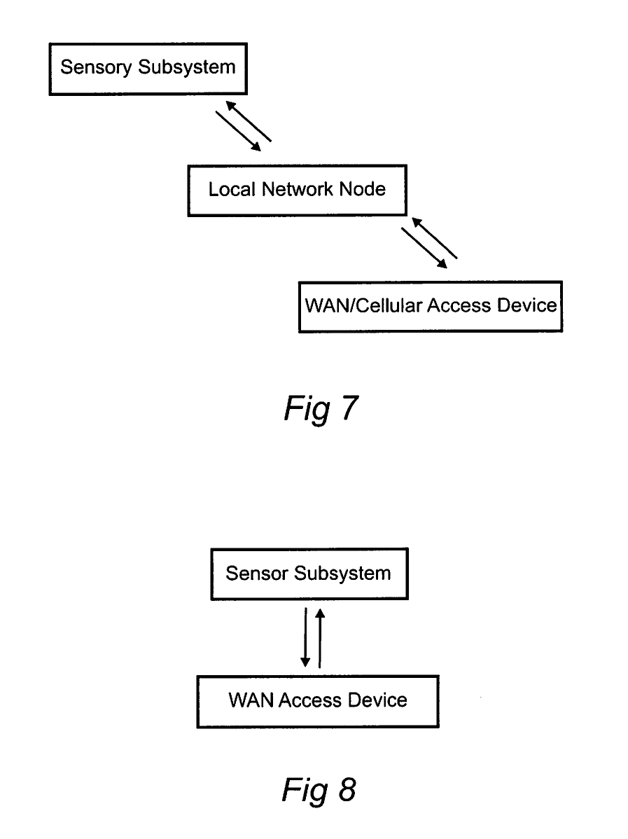

FIG. 7 shows a connectivity diagram for an embodiment of a system in accordance with the present disclosure for monitoring a surgical site. The system includes a sensory subsystem, which may be part of or an embodiment of an elongate sensing subsystem or an elongate probe in communication with a local network node, which may be a communication subsystem as discussed previously. The local network node may further be in communication with a wide area network (WAN) access device. In some embodiments, the communication subsystem may provide the functionality of a WAN access device. Additionally or alternatively, the communication subsystem may be configured to communicate with a WAN, LAN, cellular network, or the like.

In the FIG. 7 embodiment, the local network node establishes communication between the sensory subsystem and the WAN access device. This may have advantages in cases where the power required to maintain a direct link between the sensory subsystem and the WAN access device exceeds the power that can be realistically achieved from the sensory subsystem directly. In general, such a link may be necessary when the sensory subsystem is fully implanted into the body, especially when size constraints are placed on the sensory subsystem. Additionally or alternatively, the local network node may enable communication between two separate networks. For reasons of efficiency, it may be preferable to interface between the sensory subsystem and the local network node with a first protocol and spectrum, while communicating with the WAN access device using a second, optionally higher power or standardized protocol and spectrum.

Optionally, an embodiment may not require a WAN access device, in which case communication between the sensory subsystem and the local network node, or a communication subsystem are all that is required for the application. In practice, it may be advantageous to communicate data or alerts to a remote device, thus requiring a WAN access device.

The communication subsystem may be a network hub, a local network node, or the like, capable of establishing long range communication between the system and a data network. Additionally or alternatively, the communication subsystem may include a monitor, capable of communicating information to a user, patient, clinician and/or doctor about the state of the surgical site. The communication subsystem may also be a mobile computing device such as a mobile phone, an e-reader, a tablet, a media player, a mobile network node, or a pager. The communication subsystem may also be a customized hub for establishing communication and transmitting physiologically relevant data between the elongate sensory subsystem and a data network such as an institutional data network, a LAN, a WAN, a cellular network, or the like.

FIG. 8 shows another connectivity diagram for an embodiment of a system for monitoring a surgical site in accordance with the present disclosure. In the embodiment shown in FIG. 8, the sensing subsystem is provided in direct communication with a WAN access device. In this case, the WAN access device is a communication subsystem as previously described. This arrangement can be preferable in terms of reducing the number of components required to monitor the surgical site.

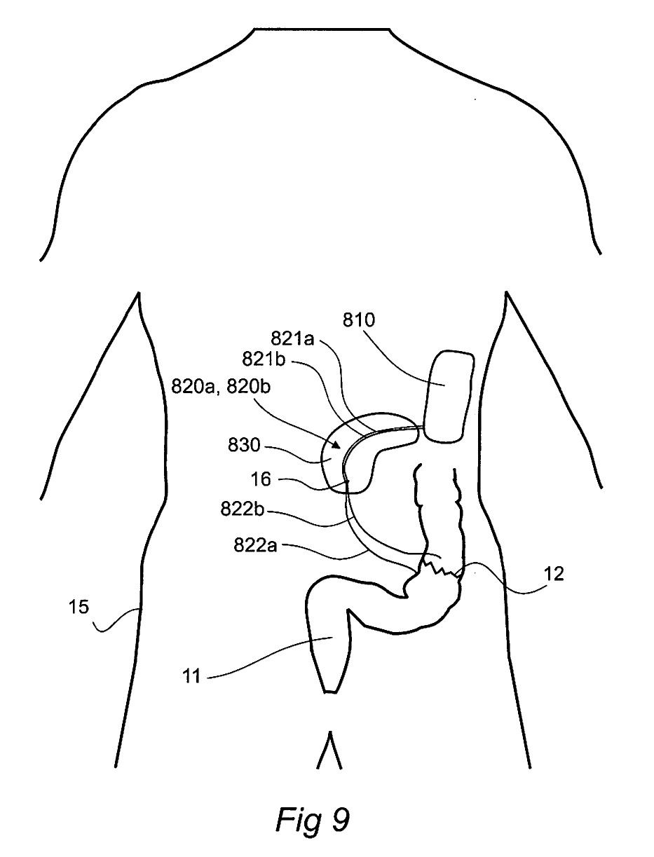

FIG. 9 schematically shows another embodiment of a system in accordance with the present disclosure for monitoring a surgical site 12, on or in an organ 11 within a body 15, including one or more elongate probes 820a, b. In this case, for the purposes of illustration, two elongate probes 820a, b are shown. The system also includes a communication subsystem 810 adapted for monitoring the surgical site 12 (e.g. a gastrointestinal anastomosis). In general, the communication subsystem 810 is located externally to the body 15 but it may also be implanted within the body 15. The elongate probe 820a, b is connected to the communication subsystem 810 and generally extends from the communication subsystem to the surgical site 12. The elongate probe 820a, b may have an externally located portion 821a, b and an internally located portion 822a, b. The length and orientation of the external portion 821a, b and internal portion 822a, b are generally determined by the location of the surgical site 12 and the makeup and proportions of the body 15. The externally located portion 821a, b and the internally located portion 822a, b come together at a portal 16 through the skin of the body 15.

The external portion 821a, b of the elongate probe 820a, b along with the portal 16 may be covered with barrier film 830 to prevent infection or fluid transfer into or out of the body 15 through the portal 16 during use.

The elongate probe 820a, b may be shaped like a slender lace, tube, cord, or conduit connected to the communication subsystem 810. The elongate probe 820a, b may be highly flexible so as to move with the tissues within the body 15 so as to minimize tissue trauma during use.

The length of the elongate probe 820a, b may be adjustable during use. In practice, estimating the distance between the surgical site and an adequate attachment point for the communication subsystem can be challenging. In the case of soft tissue surgery, such as during bowel surgery, the distance may be exceptionally challenging to determine, and may change significantly during the postoperative period. In this situation, being able to non-invasively adjust the length of the elongate probe 820a, b may be beneficial for reliable use thereof.

The elongate probe 820a, b may include one or more sensors for monitoring the surgical site 12. In the case of multiple elongate probes 820a, b each elongate probe 820a may include one or more sensors, which may be the same as, or different from sensors incorporated into an elongate probe 820b. The sensor may be an elastomeric capacitive strain and/or tension sensor, an electrode-based or bioelectrode-based impedance sensor, or a sensor configured to monitor, measure or detect one or more physiological or electrophysiological properties associated with the surgical site 12. Such physiological properties may include bioelectric activity, biopotentials, bioimpedance, bioimpedance tomography, motility, water content, pH, ionic strength, blood perfusion, and/or tissue oxygenation. The sensor may also be configured to monitor, measure or detect one or more disease states associated with the surgical site 12 including ischemia, phagocytosis related to necrotic tissue consumption, inflammation, apoptosis, progression of wound healing, abscess formation, edema, a tear, and/or onset and/or development of a leak. The sensor may further be configured to detect the presence of or measure concentrations of an analyte at the surgical site 12 including hemoglobin, lactases, glycerin, water, oxygen, and/or matrix metalloproteinases. The sensor may include, without limitation, an amperometric sensor, a potentiometer, a potentiostat, a bipotentiostat, a polypotentiostat, enzyme catalysis based sensors, a redox cell, a biofunctionallized ion-selective field effect transistor, an impedance spectrometer, or a potentiometric biosensor. Furthermore, the sensor may include, without limitation, an accelerometer, a piezoelectric sensor, a gyroscope, a pressure sensor, an EKG sensor, an EMG sensor, a temperature sensor, a pH sensor, a glucose sensor and/or an acoustic sensor. The sensor may also include, without limitation, a diffuse reflectance spectroscope, near infrared spectroscope, visible light spectroscope, surface plasmon resonant sensor, fluorescence spectroscope, ultra violet spectroscope, micro total analysis system, lab on a chip, dual or multi-wavelength biosensor, miniaturized evanescent wave biosensor, pulsed oximeter, or the like to monitor the surgical site 12. Furthermore, a plurality of sensors may be incorporated into the elongate probe 820a, b wherein the plurality of sensors may be arranged in an array to better image or map a property in and/or around a surgical site 12.

The elongate probe 820a, b may be inserted into the body 15 and fastened or placed near the surgical site 12 prior to, during or following a surgical procedure. The elongate probe 820a, b generally exits the body through a portal 16. The portal 16 may be formed separately from a surgery, or may be a laparoscopic surgical port, an entry point, or the like, formed during a surgical procedure. The elongate probe 820a, b may also be placed along with or through the same portal 16 as another surgical device such as a surgical drain.

In order to minimize tissue trauma during insertion and use, the elongate probe 820a, b may have an elastic modulus of less than 200 MPa, less than 75 MPa, less than 10 MPa, or less than 5 MPa. By elastic modulus is meant the overall elastic modulus as measured during a tensile test along the elongate direction of the elongate probe 820a, b. Tissue trauma during the monitoring process may be reduced by making the elongate probe 820a, b as soft and pliable as possible. The elongate probe 820a, b may be primarily constructed from a silicone elastomer such as poly(dimethylsiloxane), viscoelastic gel, collagen, a porous core elastomer, a perfluoropolyether, a silicone-containing polyurethane, a sufficiently soft polyurethane, PFPE-PDMS block copolymers, polyisoprene, polybutadiene, and/or fluoroolefin-based fluoroelastomers, an elastic protein such as resilin, and the like.

The elongate probe 820a, b may include a soft polymeric, elastomeric, or gel coating selected from the group consisting of an anti-thrombogenic coating, a non-fouling coating or an anti-bioadhesive coating. Non-limiting examples of suitable polymers may include slow ion release polymers capable of releasing silver ions, carbon, platinum, silver sulfadiazine, chlorhexidine, cadexomer iodine, chlorhexidine gluconate, polyhexamethylene biguanide, antimicrobial peptides, and graphene. Such materials may be provided by Semprus Biosciences, Hydromer, Johnson and Johnson, Arrow International, Cook Critical Care, DSM Biomedical and Edwards Lifesciences among others. Seprafilm™ (Genzyme Corporation) may additionally or alternatively be utilized for an anti-bioadhesive coating.

The sensor may be attached to the distal end of elongate probe 820a, b. In this case, the sensor is less than 5 mm, less than 2 mm, less than 0.75 mm, or less than 0.25 mm in length and configured to interface with the surgical site 12 or tissues in the vicinity of the surgical site 12. The sensor may be in communication with the communication subsystem 810 via the elongate probe 820a, b and/or via wireless communication protocols.

The sensor may be electrically and/or mechanically interfaced with the proximal end of the elongate probe 820a, b. In this case, the sensor may interface with the surgical site 12 via the elongate probe 820a, b. Such configurations may be advantageous for systems including a reusable part such as the communication subsystem 810 and a disposable part such as the elongate probe 820a, b.

The communication subsystem 810 may be adhesively attached to the body 15 as shown. Alternatively it may be attached to an article of clothing such as a belt, furniture such as a bed, placed in a pocket, or held in a hand.

The communication subsystem 810 may be a network hub, a local network node, or the like, capable of establishing long range communication between the system and a data network. Additionally or alternatively, the communication subsystem 810 may include a monitor, capable of communicating information to a user, patient, clinician and/or doctor about the state of the surgical site 12. The communication subsystem 810 may also be a mobile computing device such as a mobile phone, an c-reader, a tablet, a media player, a mobile network node, or a pager. The communication subsystem 810 may also be a customized hub for establishing communication and transmitting physiologically relevant data between the elongate sensory subsystem and a data network such as an institutional data network, a LAN, a WAN, a cellular network, or the like.

The communication subsystem 810 may include a sensor communication component that establishes and maintains communication with the elongate probe 820a, b and any sensors therein. Alternatively, additionally, or in combination with the sensor communication component, the communication subsystem 810 may further include a radio and antenna to communicate with the elongate probe 820a, b as well as any sensors included therein, and/or to communicate with an external network. The communication subsystem 810 may also include, without limitation, a processor, memory, one or more clocks, a signal conditioning front end, a micropump, a controller, one or more valves and/or one or more reservoirs. The communication subsystem 810 may also include one or more secondary sensors including, without limitation, a temperature sensor, EKG sensor, EMG sensor, humidity sensor, altimeter, accelerometer, a piezoelectric sensor, a gyroscope, a pressure sensor, a pH sensor, a glucose sensor or an acoustic sensor.

The physiological data communicated between the communication subsystem 810 and the elongate probe 820a, b may include, without limitation, raw data, metrics generated from the raw data pertaining to the progression or state of a surgical site 12, general patient information, device identification information, calibration data, vital sign data or metrics formulated from the vital sign data, alerts, information about patient movement, information pertaining to trauma of the surgical site 12, or the like.

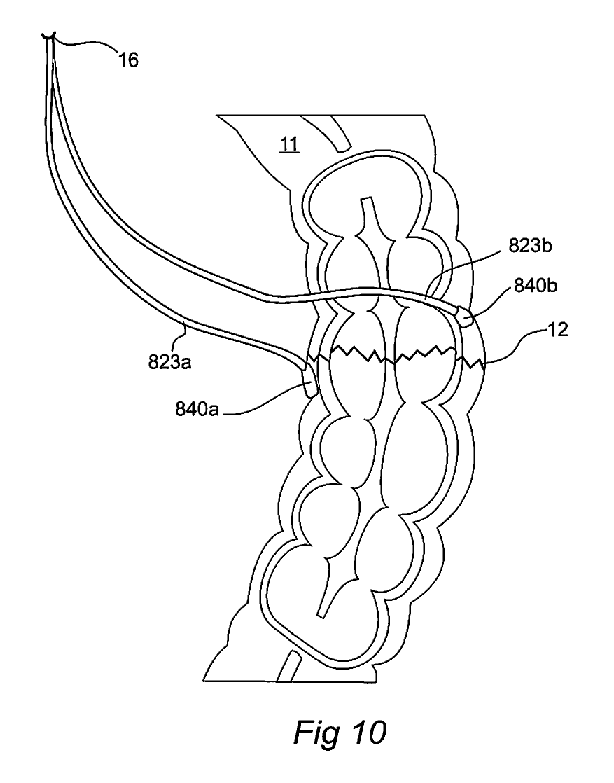

FIG. 10 shows an embodiment of a system in accordance with the present disclosure for monitoring a surgical site 12 on or in an organ 11 including two elongate probes 823a, b with bioadhesives 840a, b placed at the distal ends for retaining the elongate probes 823a, b in close vicinity to or at the surgical site 12 (e.g. a gastrointestinal anastomosis). The number of elongate probes 823a, b may be more or less than two, although two elongate probes 823a, b are shown for purposes of discussion. As discussed previously, the bioadhesives 840a, b primarily bond, in a permanent or temporary fashion, the elongate probe 823a, b to tissues near or at the surgical site 12. In this embodiment, the elongate probes 823a, b enter the body through a portal 16.

Bioadhesives are generally biocompatible adhesives that can suitably bond the elongate probe 823a, b to tissues near a surgical site 12. Bioadhesives may be non-toxic, non-fouling and biocompatible so as to help minimize the foreign body response during the monitoring process. Some suitable bioadhesives may include without limitation, polysiloxanes, polyacrylates, polyisocyanate macromers or mixtures, fibrin sealants, albumin glue with gluteraldehyde as crosslinker, hydrogels such as those formed from chitosan and poly(ethylene glycol), gelatin based adhesive with resorcinol-formaldehyde complex, oxidized polysaccharides with water-dispersible, multi-arm polyether amine, among others. The bioadhesives as noted above may also be sufficiently stable so as to retain the elongate probe 823a, b near to the surgical site 12 during the postoperative recovery period but yet sufficiently biodegradable such that retention is only maintained for a reasonable period of time, e.g. 3 weeks, 2 weeks, 1 week, 3 days, 1 day, 12 hours, 3 hours or 1 hour. In the case of a gastrointestinal anastomosis 12, the elongate probe 823a, b may be retained for up to 3 weeks, 2 weeks or 1 week.

The bioadhesives 840a, b may also electrically interact with the surgical site 12. To achieve this interaction, the bioadhesives 840a, b may be electrically or ionically conductive such as provided by ionically conductive hydrogels including dispersions of water soluble salts, silver chloride, and the like. The bioadhesive may also be part of a high impedance electrode, capable of obtaining high frequency interactions with adjacent tissues while forgoing DC interactions. A non-limiting example of a high impedance electrode material is EXH 585 adhesive available from FLEXcon Company Inc. of Spencer, Mass. Other non-limiting examples include conductive carbon coatings, intrinsically conducting polymers such as Poly(3,4-ethylenedioxythiophene) (PEDOT) available commercially as Clevios™, carbon nanotube dispersions such as Super HiPCO™ from Carbon Nanotechnologies Inc. of Houston, Tex. In addition, the bioadhesives may be provided in the form of an ion-conducting membrane so as to provide fluid exchange across the bioadhesive region 840a, b in addition to adhesive capabilities in a single structure.

Monitoring of the surgical site 12 may be achieved via sensors or electrode elements placed within one or more elongate probes 823a, b. In addition, multiple elongate probes 823a, b such as those shown may be employed in a coordinated manner to monitor the surgical site 12. In this case, one or more elongate probes 823a may include one or more electrodes, light sources, photodiodes and/or similar sensors configured so as to interact with sensors, electrodes and/or optical fibers provided in one or more elongate probes 823b. Such interactions may take place across or near to the surgical site 12 thus allowing sent and received signals to be processed by coordinated interaction of multiple elongate probes 823a, b.

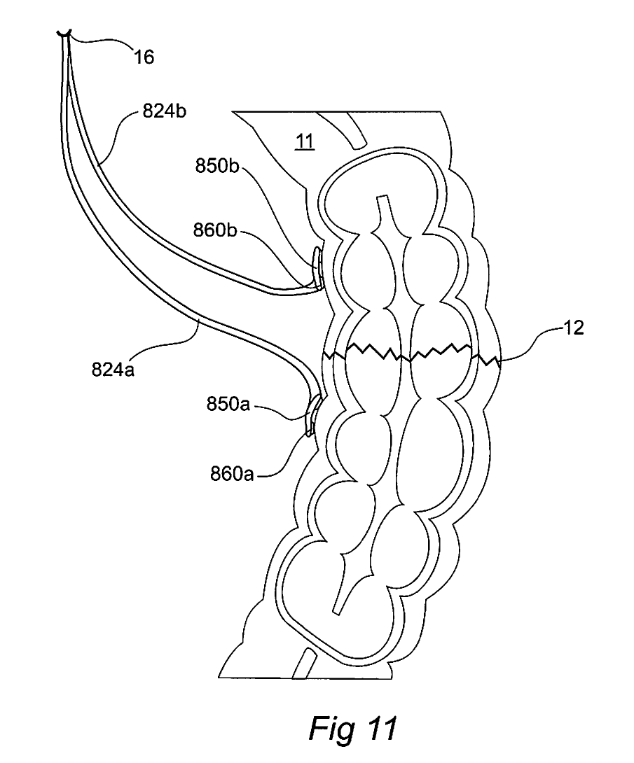

FIG. 11 shows another embodiment of part of a system for monitoring a surgical site 12 in accordance with the present disclosure including one or more elongate probes 824a, b. In this case, two probes 824a, b each with one or more electrode sets 860a, b are configured so as to monitor tissues of an organ 11 (e.g. an intestine) on both sides of a surgical site 12 (e.g. a gastrointestinal anastomosis). In this embodiment, the elongate probes 824a, b enter the body through a portal 16.

The elongate probes 824a, b may also include elastic or elastomeric electrically conducting elements configured to provide electrical interfacing between a communication subsystem (not explicitly shown) and the electrode sets 860a, b. In this embodiment, the system may include one or more soft polymeric or elastomeric conducting fibers arranged along the length of the elongate probe 860a, b to provide electrical interfacing between the communication subsystem (not explicitly shown) and the surgical site 12 via the electrode sets 860a, b. One configuration may include a composite formed from a non-conducting elastomer such as a silicone or polyurethane and one or more electrically conducting filler materials such as metallic or carbon powders, or conjugated polymer fillers such as PEDOT doped with poly(styrenesulfonate) (PEDOT/PSS). Additionally or alternatively, a composite structure may be used to couple a thin film non-elastomeric conductor with an elastomeric substrate to form an equivalent to an elastomeric conducting fiber. In this case, the thin film non-elastomeric conductor is deposited on the surface of a stretched or preformed elastomeric substrate so as to form a series of buckled structures in operation. The buckled structures allow for the entire composite to stretch without damaging the non-elastomeric conductors. Stretchable electronic interconnections to connect rigid semiconducting islands may also be employed. The electronic interconnections may be provided by meandering thin-film traces and/or by lift off of thin-film traces from an elastomeric substrate such as silicone. Such an approach can also be used to create an equivalent to an elastomeric conducting fiber.

The electrode sets 860a, b may be attached to flexible supports 850a, b that interconnect with the elongate probes 824a, b. The flexible supports 850a, b may be sufficiently soft so as to allow movement of the electrode sets 860a, b with the tissues of the attached organ 11 during movements. Such movements may be due to local muscle contractions, bodily movements, postural changes, or the like.

The electrode sets 860a, b and/or the flexible supports 850a, b may include a bioadhesive as described previously.

Additionally or alternatively, the elongate probes 824a, b may include one or more elastomeric optical fibers adapted so as to provide optical (e.g. visible light, near infrared light, etc.) communication between the distal and proximal ends of the probes 824a, b. The flexible supports 850a, b may include interfaces for emitting light or receiving light with the embedded elastomeric optical fibers.

The elongate probe 824a, b may be retractable or adjustable in length such that the distal end of the probe 824a, b can be easily maintained near to the surgical site 12 without requiring an inventory of several different probes 824a, b.

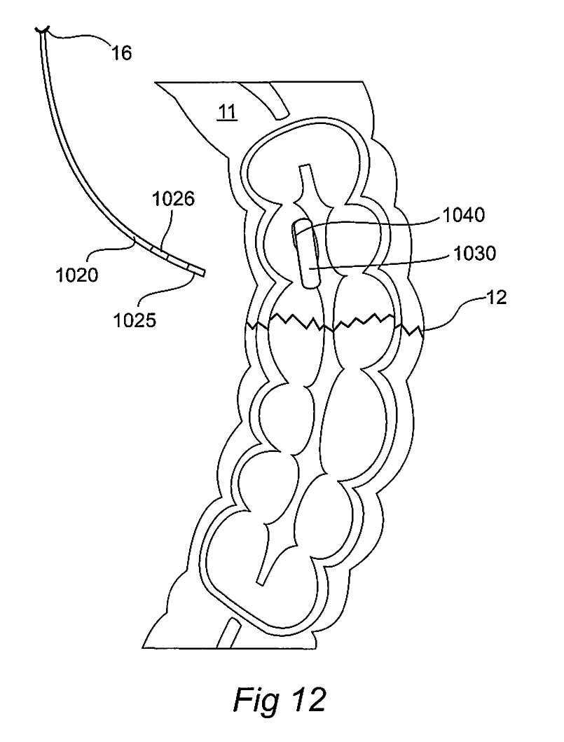

FIG. 12 shows an embodiment of part of a system for monitoring a surgical site 12 in accordance with the present disclosure including an elongate probe 1020 placed externally to an organ 11 and an elongate sensing subsystem 1030 placed internally to the organ 11 relatively near to a surgical site 12 (e.g. a gastrointestinal anastomosis). The elongate probe 1020 may be retractable or adjustable in length so as to maintain its position and/or orientation relative to the organ 11 and/or the surgical site 12 during use. The sensing subsystem 1030 may be attached to the organ 11 near to or at the surgical site 12 using an attachable region 1040. The attachable region 1040 may include a bioadhesive, such as, without limitation, a biodegradable bioadhesive, a mechanical interfacing mechanism, a structure (e.g. an eyelet, loop, hook, etc.) for receiving a suture, a hook type fastener, barbed fastener, or the like to retain the elongate sensing subsystem 1030 to the organ 11 or surrounding tissues. An attachable region 1040 including hooks and/or barbs can be a particularly advantageous way to interface with the surgical site 12 in that the hooks and/or barbs can be constructed so as to conduct electricity as well as be made sufficiently biodegradable that the fixation points will weaken and break away from the body after a sufficiently long period of interaction. In one embodiment, the attachable region 1040 may be amendable to receiving one or more sutures. Acceptable sutures may be formed from cotton, silk, nylon and/or polypropylene among other common materials. The suture materials may be biodegradable including, without limitation, such materials as surgical gut, chromic suture materials (generally type A or Type B), polyglycolic acid, polylactic acid, polydioxanone, caprolactone amongst others.

The attachable region 1040 may also include a bioadhesive as previously described.

The elongate probe 1020 may reside partially within the body and partially outside the body extending through a portal 16 in the skin wall of the body.

The elongate probe 1020 may include a series of sensors, light sources, photodiodes, or the like for monitoring the adjacent tissues and/or interacting with the elongate sensing subsystem 1030. The elongate probe 1020 may also include one or more electrodes 1025, 1026 arranged near to the distal end of the probe 1020 to electrically interface with the surroundings and/or the elongate sensing subsystem 1030.

The elongate probe 1020 and the elongate sensing subsystem 1030 may interact with each other via the tissues in and around the surgical site 12 and surrounding organ 11 so as to assess the progression of the surgical site 12. In one embodiment, the elongate probe 1020 includes one or more electrodes 1025 1026 that can sense and respond to an ultrasonic or RF pulse provided by the sensory subsystem 1030. Additionally or alternatively, the sensory subsystem 1030 can detect a pulse or pulse train provided by the elongate probe 1020. The signals may be reflected by either source with gain modulation proportional to the strength of the received signal. Generally, signal strength and phase will change due to both proximity and orientation of the probe electrodes 1025, 1026 and the elongate sensing subsystem 1030 as well as the integrity of the walls of the organ 11 and/or the surgical site 12. Furthermore, the bioelectric response to an ultrasonic excitation made by either the elongate probe 1020 or the elongate sensory subsystem 1030 may provide advantageous data regarding progression of the surgical site 12 and surrounding tissues. In this way, the coordinated effort of the elongate probe 1020 and the elongate sensory subsystem 1030 can be used to assess the properties of the organ 11 and particularly of the surgical site 12 located there between.

The signals may be a pulse train, chirp, decaying chirp, adaptive signals, or the like. The signals may be for example, RF or ultrasonic signals. In the case of an ultrasonic signal, the elongate probe 1020 and/or the elongate sensing subsystem 1030 may include a piezoelectric, MEMs based, capacitive, and/or electromagnetic actuator suitable of producing the aforementioned signals. The elongate sensing subsystem 1030 and/or the elongate probe 1020 may include a sensory system for detecting the emitted signal. In this case, either one may include a piezoelectric, piezoresistive, hybrid modular detectors, capacitive, MEMs, or electromechanical detection means. Additionally or alternatively, the signal generator and receiver may include a microspeaker and a microphone, respectively.

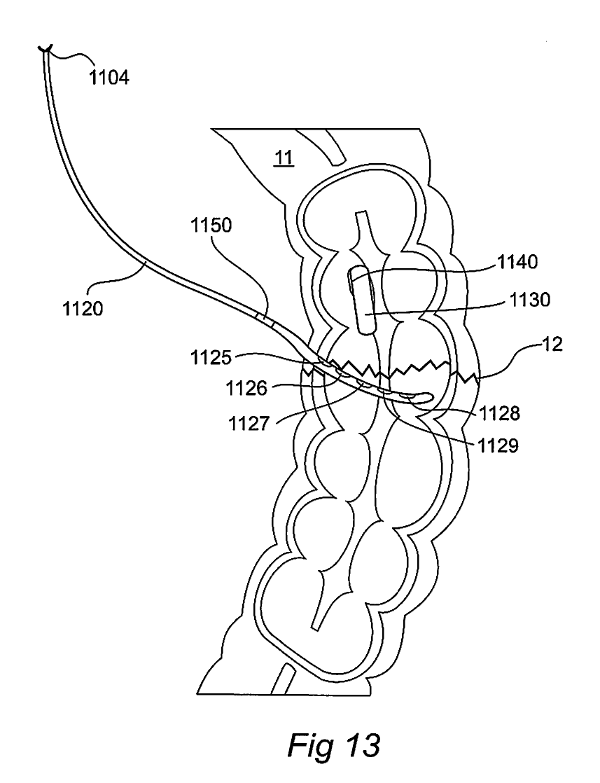

FIG. 13 shows a portion of an alternative embodiment of a system for monitoring a surgical site 12 inside a body including an elongate probe 1120 and optionally an elongate sensory subsystem 1130 adapted for placement internally to the organ 11. The elongate probe 1120 includes multiple active electrodes 1125-1129 and optionally one or more reference electrodes 1150 attached to the outer wall of the elongate probe 1120 near to the distal end of the elongate probe 1120. The electrodes 1125-1129 and the counter electrode 1150 may be draped along the outside wall of the organ 11 and an elongate sensing subsystem 1130 placed within the organ 11 (e.g. an intestine) near to the surgical site 12 (e.g. an anastomosis). The sensory subsystem 1130 may be fastened to the organ 11, preferably near to or at the surgical site 12 using an attachable region 1140 with similar properties and material options as previously discussed.

The electrodes 1125-1129 may be generally configured to interface with the tissues around a surgical site 12, the organ 11, the fluids in the vicinity of the surgical site 12, or the tissues within the surgical site 12. The electrodes 1125-1129 may be at least partially formed from an electrically conducting material such as, without limitation, platinum, silver, silver/silver chloride, carbon materials (e.g. activated carbon, graphite, graphene, etc.), gold, intermetallics, stainless steel, and the like. The electrodes 1125-1129 may further include a radiopaque (e.g. radiodense) material such as, without limitation, platinum, gold, zirconium, palladium-based alloys, tungsten, tantalum, and the like. Such radiopaque materials may be advantageous for imaging placement of electrodes 1125-1129 in vivo. Additionally or alternatively, the electrodes 1125-1129 may include titanium nitride or cermets as a thin protective biocompatible layer. In embodiments, the electrodes may be formed from a printable ink including a binder and conducting filler. The electrodes may further include a conjugated polymer such as, without limitation, PEDOT, polyanaline, polypyrrole, polythiophenes, polyacetylene, and the like. The electrodes 1125-1129 may include a radio opaque component so as to improve visualization with various imaging methods. The electrodes 1125-1129 may also be insulated bioelectrodes generally including a conducting electrode material with a thin dielectric overcoat. The dielectric overcoat may improve long term stability of the electrodes when placed in contact with living tissues. The reference electrode 1150 may be at least partially formed from any of the above materials but may also be at least partially formed from a stable material such as silver/silver chloride. In one embodiment, all electrodes 1125-1129 and the reference electrode 1150 may be formed from the same material.

The electrodes 1125-1129 may be adapted so as to provide and sense signals both amongst each other and to/from the elongate sensing subsystem 1130. In one embodiment, the electrodes 1125-1129 or the elongate probe 1120 may sense and respond to an ultrasonic or RF pulse provided by the sensory subsystem 1130. Additionally or alternatively, the sensory subsystem 1130 may detect a pulse or pulse train provided by one or more of the electrodes 1125-1129. The signals may be generated by current or charge to any pair of the electrodes 1125-1129 or between any electrode 1125-1129 and the reference electrode 1150. The signals may be reflected to either source with gain modulation proportional to the strength of the received signal. Generally signal strength and phase will change due to both proximity and orientation of the probe electrodes 1125-1129, the reference electrode 1150, and the elongate sensing subsystem 1130 as well as the integrity of the walls of the organ 11 and/or the surgical site 12.

In some embodiments, the elongate sensory subsystem 1130 and/or the elongate probe 1120 may include an ultrasonic source in addition to the electrodes 1125-1129. The bioelectric response to an ultrasonic excitation made by either the elongate probe 1120 or the elongate sensory subsystem 1130 may provide advantageous data regarding progression of the surgical site 12 and surrounding tissues. In this way, the coordinated effort of the elongate probe 1120 and the elongate sensory subsystem 1130 can be used to assess the properties of the organ 11 and particularly of the surgical site 12 there between.

The signals may be a pulse train, chirp, decaying chirp, adaptive signals, or the like. The signals may be, for example, RF or ultrasonic signals. In the case of an ultrasonic signal, the elongate probe 1120 and/or the elongate sensing subsystem 1130 may include a piezoelectric, MEMs based, capacitive, and/or electromagnetic actuator suitable of producing the aforementioned signals. The elongate sensing subsystem 1130 and/or the elongate probe 1120 may include a sensory system for detecting the emitted signal. In this case, either one may include a piezoelectric, piezoresistive, hybrid modular detectors, capacitive, MEMs, or electromechanical detection means. Additionally or alternatively, the signal generator and receiver may include a microspeaker and a microphone respectively.

Coordination of multiple electrodes 1125-1129 optionally in combination with the sensory subsystem 1130 may help to localize the assessment of complications detected at the surgical site 12 or in the organ 11. Thus, such a configuration may provide more detailed information than with a single elongate probe 1120 and an elongate sensing subsystem 1130.

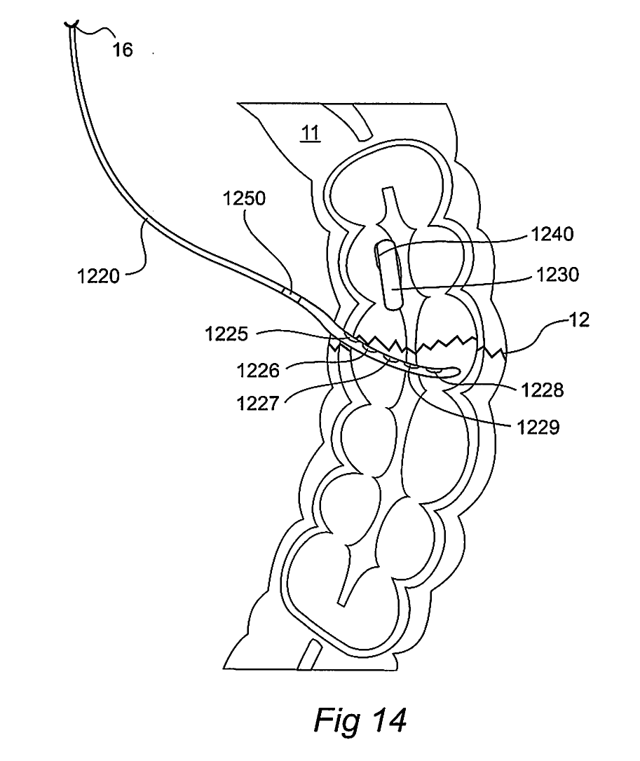

FIG. 14 shows an embodiment of a system in accordance with the present disclosure for monitoring a surgical site inside a body including an elongate probe 1220 with one or more light sources 1225, 1227, 1228 and one or more photodetectors 1226, 1229. The system also includes an elongate sensing subsystem 1230 with one or more light sources and one or more photodetectors. The elongate sensing subsystem 1230 may be placed inside of an organ 11 (e.g. an intestine) near to a surgical site 12 (e.g. an anastomosis). The sensory subsystem 1230 may be fastened to the organ 11 near to or at the surgical site 12 using an attachable region 1240 with similar properties and material options as previously discussed.

The light sources 1225, 1227 1228 and photodetectors 1226, 1229 may be configured to interface with the tissues around a surgical site 12, the organ 11, the fluids in the vicinity of the surgical site 12, or the tissues within the surgical site 12. The light sources 1225, 1227, 1228 may be laser diodes, light emitting diodes, or the like with narrowband or broadband emission spectra. The light sources 1225, 1227, 1228 may be operated in a continuous or pulsed fashion as dictated by the sensing protocol. The photodetectors 1226, 1229 may be narrow or broad band detectors, and may be configured with filters or optical elements to adjust angles at which light can be detected. Light sources and photodetectors incorporated into the elongate sensing subsystem 1230 may have the same or similar properties as those included on the elongate probe 1220.

The light sources 1225, 1227, 1228 and photodetectors 1226, 1229 may be used to measure various physiological parameters of the tissues in and near to the surgical site 12 including oxygen concentration, oxygenated hemoglobin concentration, deoxygenated hemoglobin concentration, matrix metalloproteases, and/or water content (oedema). The system may include, without limitation, a diffuse reflectance spectroscope, near infrared spectroscope, visible light spectroscope, surface plasmon resonant sensor, fluorescence spectroscope, ultra violet spectroscope, micro total analysis system, lab on a chip, dual or multi-wavelength biosensor, miniaturized evanescent wave biosensor, pulsed oximeter, or the like in communication with the light sources 1225, 1227, 1228 and photodetectors 1226, 1229 to monitor the surgical site 12.

The photodetectors 1226, 1229 may be adapted to sense signals emitted from any of the light sources 1225, 1227, 1228 or from the elongate sensing subsystem 1230. Additionally or alternatively, a photodetector embedded in the elongate sensing subsystem 1230 may be configured so as to receive light emitted by a light source embedded within the elongate sensing subsystem 1230 and/or the light sources 1225, 1227, 1228. In some instances, signal strength and/or phase lag of the signals may change due to proximity and/or orientation of light sources 1225, 1227, 1228, photodetectors 1226, 1229 and the elongate sensing subsystem 1230 as well as the integrity of the walls of the organ 11 and/or the surgical site 12.

Coordination of multiple light sources 1225, 1227, 1228 and photodiodes 1226, 1229, optionally in combination with the sensory subsystem 1230, may help to localize the assessment of complications detected at the surgical site 12 or in the organ 11. Thus, such a configuration may provide more detailed information than with a single elongate probe 1220 and an elongate sensing subsystem 1230.

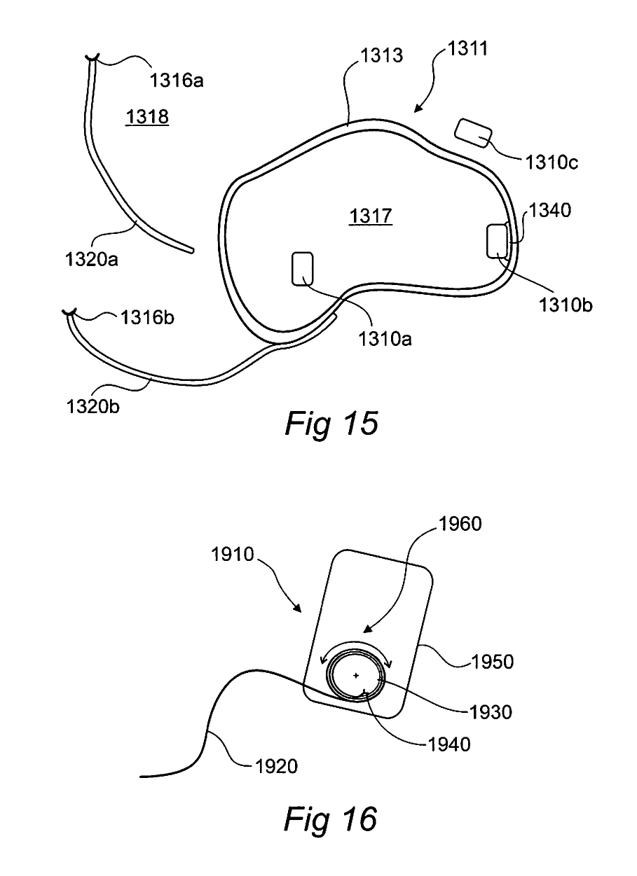

FIG. 15 shows multiple embodiments of elongate probes 1320a, b and elongate sensory subsystems 1310a, b, c arranged in the vicinity of an organ 1311. The organ may have a surgical site 1312 located internally or near to an organ wall 1313. The number and placement of elongate probes 1320a, b and elongate sensing subsystems 1310a, b, c may vary depending on the type of organ 1311, the location of the surgical site 1312 and the type of complications commonly experienced during postoperative recovery.

As shown, a first elongate probe 1320a may be inserted into the body via a portal 16a with the distal end of the elongate probe 1320a being placed in the external space 1318 near to the organ 1313. Additionally or alternatively, is shown a second elongate probe 1320b which may be inserted into the body via a portal 1316b with the distal end of the elongate probe 1320b being placed adjacent to or affixed to the organ wall 1313. Elongate probes 1320a, b may also be placed in the organ interior 1317 although an embodiment of this type is not shown.

An elongate sensory subsystem 1310a may be placed in the organ interior 1317 at or near to the surgical site 1312. Alternatively or additionally, an elongate sensory subsystem 1310b maybe placed adjacent to or affixed to the organ wall 1313. In this case, the sensory subsystem may have a means for fixation 1340 such as a bioadhesive, mechanical mechanism, and/or a means for accepting one or more sutures as previously described herein. In another non-limiting example, an elongate sensory subsystem 1310c may be placed in the external space 1318 around the organ 1311.

The elongate sensory subsystem 1310a, b, c and the elongate probe 1320a, b may include features as described throughout this disclosure.

Such elongate probes 1320a, b and/or sensory subsystems 1310a, b, c may be used individually or collectively as required by the particular surgical case at hand.

The organ 1311 may be any organ, e.g., a colon, small intestine, bile duct, pancreas, stomach, esophagus, artery, vein, ureter, urinary bladder, urethra, or nerve. Alternatively, the organ 1311 may be a kidney, liver, heart, lung, pancreas, ovaries, uterus, brain, or the like. The organ 1311 may be part of the musculoskeletal system such as a bone, ligament, tendon, cartilage, joint, or the like. The surgical site 1312 may be an anastomosis, excision of a tumor, removal of an organ or part of an organ, an implant site, reconstructive surgical site such as tendon repair, trauma site such as a bone fracture, or the like. The system may be used to monitor general healing in and around the surgical site 1312 as well to as monitor for complications such as implant rejection, thrombosis formation, thrombosis migration, infection, tear formation, leak formation, abscess formation, edema formation, ischemia, tissue degradation, foreign matter, arrhythmia, changes in organ function such as changes in motility, and the like.

FIG. 16 shows a schematic view of a communication subsystem 1910 including a mechanism 1930 for adjusting the length of an attached elongate probe 1920. In the embodiment shown, the communication subsystem 1910 includes a spindle 1930 upon which the elongate probe 1920 may be wound so as to retract or advance the elongate probe 1920 during use. The elongate probe 1920 generally passes through the housing 1950 of the communication subsystem, is wound onto the spindle 1930 and interconnects with the communication subsystem via a terminal connector 1940. The terminal connector 1940 may be adapted for electrically interfacing between the elongate probe 1920 and the communication subsystem 1910. Alternatively or additionally, the terminal connector 1940 may be adapted to provide light communication between the elongate probe 1920 and the communication subsystem 1910.

In addition, the spindle 1930 may include signal conditioning, data acquisition, power management, light sources/receivers, and/or communication circuitry adapted to relay information collected by the elongate probe 1930 to other electronics within the communication subsystem 1910. These elements may be used to ensure minimal signal corruption that can occur with multiple connectors between the elongate probe 1920 and the communication subsystem 1910.

The communication subsystem 1910 may include a motor or equivalent for winding the spindle 1930. In this case, the communication subsystem 1910 may automatically advance or retract the elongate probe 1920 in the directions 1960 indicated. The communication subsystem 1910 may include a manual dial, button, crank, or the like such that a user may conveniently advance or retract the elongate probe 1920 during use.

Alternatively to the embodiment shown, the mechanism 1930 may be a sliding track, a folding mechanism, an external track that may be adjusted by rotating the communication subsystem 1910, or the like for adjusting the length of the elongate probe 1920 extending from the communication subsystem 1910.

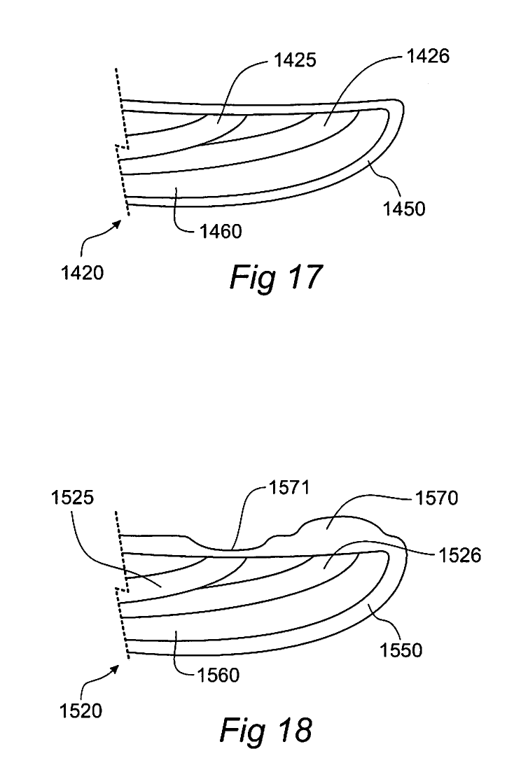

FIG. 17 shows a cross section of an embodiment of a distal end of an elongate probe 1420 with embedded elastomeric optical fibers 1425, 1426 configured to deliver light between a communication subsystem and a surgical site. The elongate probe 1420 further includes an elastomeric matrix 1460 into which the elastomeric optical fibers 1425, 1426 are embedded. In addition, an optional biocompatible overcoat 1450 is provided so as to isolate the optical fibers 1425, 1426 and the matrix 1460 from the surroundings. Although only two elastomeric optical fibers 1425, 1426 are shown, an embodiment may include more or less fibers 1425, 1426 as needed for a given application.

The elongate probe 1420 may be retractable. The elastomeric optical fibers 1425, 1426 are generally arranged so as to transmit light even under stretch. A soft elongate probe 1420 may be important for minimizing local tissue trauma during use. The primary mechanical properties of the elongate probe 1420 are determined by the constituents. The elastomeric matrix 1460 may be primarily constructed from a silicone elastomer such as, without limitation, poly(dimethylsiloxane), viscoelastic gel, collagen, a porous core elastomer, a perfluoropolyether, a silicone-containing polyurethane, a sufficiently soft polyurethane, PFPE-PDMS block, polyisoprene, polybutadiene, and/or fluoroolefin-based fluoroelastomers, an elastic protein such as resilin, and the like.

The overcoat 1450 may be formed from a soft polymeric, elastomeric, or gel selected from the group consisting of an anti-thrombogenic coating, a non-fouling coating or an anti-bioadhesive coating. Non-limiting examples of suitable polymers may include, without limitation, slow ion release polymers capable of releasing silver ions, carbon, platinum, silver sulfadiazine, chlorhexidine, cadexomer iodine, chlorhexidine gluconate, polyhexamethylene biguanide, antimicrobial peptides, and graphene. Such materials may be provided by Semprus Biosciences, Hydromer, Johnson and Johnson, Arrow International, Cook Critical Care, DSM Biomedical and Edwards Lifesciences among others. Seprafilm™ (Genzyme Corporation) may additionally or alternatively be utilized for an anti-bioadhesive coating.

The elastomeric optical fibers 1425, 1426 may be configured to transmit light along the elongate probe between the communication subsystem and the surgical site. Elastomeric optical fibers 1425, 1426 allow for the transfer of light to or from the surgical site through the elongate probe 1420. Elastomeric optical fibers 1425, 1426 may be formed from highly pure silicone elastomers. The elastomeric optical fibers 1425, 1426 may be embedded into the elongate probe 1420 as shown. Alternatively or additionally, the elastomeric optical fibers 1425, 1426 may be provided separately running alongside the elongate probe 1420. In addition, elastomeric optical fiber 1425, 1426 may be arranged within a lumen of the elongate probe 1420.

In combination with elongate probe 1420 including elastomeric optical fibers 1425, 1426 the monitoring system may include one or more light sources for emitting light towards the surgical site and one or more photodetectors for receiving light from the surgical site. Generally light is transmitted to and from the surgical site using the elastomeric optical fibers 1425, 1426. In combination with the fibers 1425, 1426 the light sources and photodetectors may be used to measure various physiological parameters of the tissues in and near to the surgical site including oxygen concentration, oxygenated hemoglobin concentration, deoxygenated hemoglobin concentration, matrix metalloproteases, and/or water content (edema). The system may include, without limitation, one or more diffuse reflectance spectroscope, near infrared spectroscope, visible light spectroscope, surface plasmon resonant sensor, fluorescence spectroscope, ultra violet spectroscope, micro total analysis system, lab on a chip, dual or multi-wavelength biosensor, miniaturized evanescent wave biosensor, pulsed oximeter, or the like in communication with the light source and photodetector to monitor the surgical site.

FIG. 18 shows a cross section of a distal end of an elongate probe 1520 including elastomeric optical fibers 1525, 1526 embedded in an elastomeric matrix 1560. In this embodiment the figure shows a cross section of the tip of an elongate sensing subsystem. The elongate probe 1520 is further coated with a biocompatible coating 1550 including lenses 1570, 1571 at the tips of the optical fibers 1525, 1526. The lenses 1570, 1571 are optionally formed from the same material as the biocompatible coating 1550. The lenses 1570, 1571 are generally shaped so as to create a diverging, converging and/or redirected path for light passing from the optical fibers 1525, 1526 to the surgical site or vice versa. Lenses 1570, 1571 may be formed from material that is transparent, translucent, and/or combinations thereof. The lenses 1570, 1571 may have a convex, concave, diffraction grating, or similar construct or texture so as to achieve the desired optical function. Furthermore, the lenses 1570, 1571 may include an antireflective coating to improve the efficiency of light transmission to and from the surgical site.

The biocompatible coating 1550, elastomeric optical fibers 1525, 1526, and the elastomeric matrix 1560 may be generally formed from the materials as previously described herein.

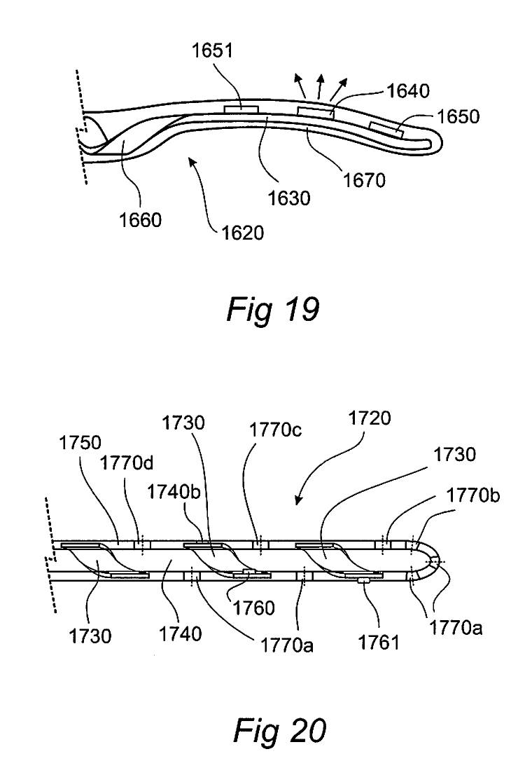

FIG. 19 shows the cross section of an embodiment of the distal end of an elongate probe 1620 or alternatively of the tip of an elongate sensing subsystem. The elongate probe 1620 or alternatively elongate sensing subsystem includes a substrate 1630 for electrical communication between the distal end of the elongate probe 1620 and the communication subsystem. The substrate 1630 may be a soft elastomeric flexible substrate as discussed previously. The substrate 1630 may also be a helically shaped flexible substrate or equivalent structure that is made flexible through twisting the substrate 1630 into a tortuous path.

The elongate probe 1620 may include a light source 1640 and two photodetectors 1650, 1651 for interfacing with a surgical site. Although only one light source 1640 and two photodectors 1650, 1651 are shown, more light sources and/or more or less photodetectors can be used as needed for the intended application. The elongate probe 1620 may also include one or more microcircuits to interface with the source 1640 and/or photodiodes 1650, 1651. The substrate 1630 may include electrically conducting traces (not explicitly shown) for interconnecting the source 1640, microcircuit and/or photodetectors 1650, 1651 with the communication subsystem.

The substrate 1630, source 1640 and photodiodes 1650, 1651 may be isolated from the surrounding body by an elastomeric biocompatible overcoat 1670 formed from materials as previously described herein.

The elongate probe 1620 and in particular the substrate 1630 may include one or more soft polymeric or elastomeric conducting fibers arranged along the length of the elongate probe 1620 or substrate 1630 to provide electrical communication between the communication subsystem and the surgical site. Additionally or alternatively, a composite structure may be used to couple a thin film non-elastomeric conductor with an elastomeric substrate to form an equivalent to an elastomeric conducting fiber. In this case, the thin film non-elastomeric conductor is deposited on the surface of a stretched or preformed elastomeric substrate so as to form a series of buckled structures in operation. The buckled structures allow for the entire composite to stretch without damaging the non-elastomeric conductors. Stretchable electronic interconnections may be employed to connect rigid semiconducting islands. Such an approach can also be used to create an equivalent to an elastomeric conducting fiber.

FIG. 20 shows a cross section of another embodiment of the distal end of an elongate probe 1720 including helical electrical interconnects 1730 embedded within, and coaxial to, the elongate probe 1720. The elongate probe 1720 may include a conduit 1740 for transferring fluids between the proximal and distal ends of the elongate probe 1720. Furthermore, the elongate probe 1720 may further include and elastomeric wall or topcoat 1750 to give shape to the probe 1720. The topcoat 1750 may be formed from a biocompatible material similar to those previously discussed.

The electrical interconnects 1730 may include one or more sensors 1760 such as those previously described. The electrical interconnects 1730 may include one or more electrodes 1761 for interfacing with the surgical site. The electrical interconnects 1730 may also include one or more microcircuits for interfacing with the sensors and/or electrodes, providing local power management, managing communication between the communication system and the distal end of the elongate probe 1720, or the like.

The elongate probe 1720 may further include a series of ports 1770a-d which form fluid paths between the conduit 1740 and the surrounding tissues and fluids. The conduit 1740 may then provide a means for collecting samples or delivering medicament from or to the surgical site.

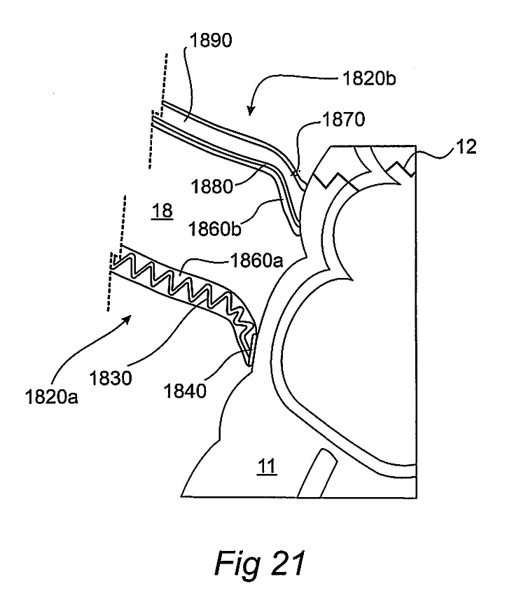

FIG. 21 shows the distal ends of two embodiments of elongate probes 1820a1820b in contact with an organ 11 (e.g. an intestine) adapted so as to monitor a surgical site 12 (e.g. an anastomosi). The first elongate probe 1820a includes an embedded spring like conducting element 1830 for electrically communicating between the proximal and distal ends of the elongate probe 1820a. The elongate probe 1820a may additionally or alternatively include an electrode and/or adhesive tip 1840. The elongate probe 1820a may also include an elastomeric matrix 1860a arranged along its length or applied as a coating. The elastomeric matrix 1860a may be formed from an elastomer or additionally or alternatively from an anti-thrombogenic coating, a non-fouling coating or an anti-bioadhesive coating. Some suitable anti-thrombogenic coatings, non-fouling coatings or anti-bioadhesive coatings have been discussed earlier.

The elastomeric matrix 1860a of the elongate probe 1820a may be formed from an elastomeric material. A suitable elastomeric material may have an elastic modulus of less than 200 MPa, less than 75 MPa, less than 10 MPa, or less than 5 MPa. By elastic modulus, it is meant the overall elastic modulus as measured during a tensile test along the elongate direction of a formed elongate probe 1820a. Tissue trauma during the monitoring process may be reduced by making the elastomeric matrix 1860a as soft and pliable as possible. The elastomeric matrix 1860a may be primarily constructed from a silicone elastomer such as poly(dimethylsiloxane), viscoelastic gel, collagen, a porous core elastomer, a perfluoropolyether, a silicone-containing polyurethane, polyisoprene, polybutadiene, fluoroolefin-based fluoroelastomers, an elastic protein such as resilin, and the like.

The embedded electrically conducting spring-like element 1830 may be an electrically conducting microspring embedded longitudinally within the elongate probe 1820a to provide electrical communication between the communication subsystem and the surgical site 12. The electrically conducting microspring 1830 may be a helical coil microspring, a crimped microspring, or the like. The microspring 1830 provides a means for electrical communication between the communication subsystem and the distal end of the elongate probe 1820a and/or the surgical site 12. In one embodiment, the microspring 1830 may provide electrical interconnection for one or more sensors located in the distal tip of the elongate probe 1820a. Additionally or alternatively, the tip of the microspring 1830 may be exposed or integrated into an electrode 1840 for interfacing with the surgical site 12. The microspring 1830 may be shaped so as to be sufficiently low modulus such that it does not adversely affect the overall elastic modulus of the elongate probe 1820a.

In some embodiments, an elongate probe 1820b is configured so as to interface with the organ 11. The elongate probe 1820b includes a conduit 1890 for providing fluid communication between the proximal and distal ends of the elongate probe 1820b. The elongate probe 1820b may also include an optional aspiration feature 1870 to optionally provide fluid exchange between the conduit 1890 and the fluid 18 surrounding the organ 11. The elongate probe 1820b may also include an elastomeric electrically conducting element 1880 arranged along the elongate probe 1820boptionally along the interior of the conduit 1890 as shown. The electrically conducting element 1880 may provide electrical communication between the proximal and distal ends of the elongate probe 1820b. Suitable elastomeric electrically conducting materials for use in the electrically conducting element 1880 have been discussed previously. The system may further include a micropump (not explicitly shown), optionally integrated into the communication subsystem, that is adapted to be in fluid communication with the conduit 1890.

The conduit 1890 may be arranged down the center line of the elongate probe 1820bor it may be part of a multilumen elongate probe, whereby one or more lumens may provide bidirectional fluid communication between the reservoir, the micropump and/or the surgical site. In the case of a multilumen elongate probe, one or more lumens may provide fluid communication from the micropump and/or reservoir to the surgical site 12. One or more lumens of a multilumen elongate probe may provide fluid communication from the surgical site to the micropump and/or a reservoir.

The wall or outer layer 1860b of the elongate probe 1820b may be formed from an elastomeric material. A suitable elastomeric material may have an elastic modulus of less than 200 MPa, less than 75 MPa, less than 10 MPa, or less than 5 MPa. By elastic modulus, it is meant the overall elastic modulus as measured during a tensile test along the elongate direction of a formed elongate probe 1820b. Tissue trauma during the monitoring process may be reduced by ensuring the elastomeric matrix 1860b be soft and pliable. The elastomeric matrix 1860b may be primarily constructed from a silicone elastomer such as, without limitation, poly(dimethylsiloxane), viscoelastic gel, collagen, a porous core elastomer, a perfluoropolyether, a silicone-containing polyurethane, a sufficiently soft polyurethane, PFPE-PDMS block copolymers, polyisoprene, polybutadiene, fluoroolefin-based fluoroelastomers, an elastic protein such as resilin, and the like.

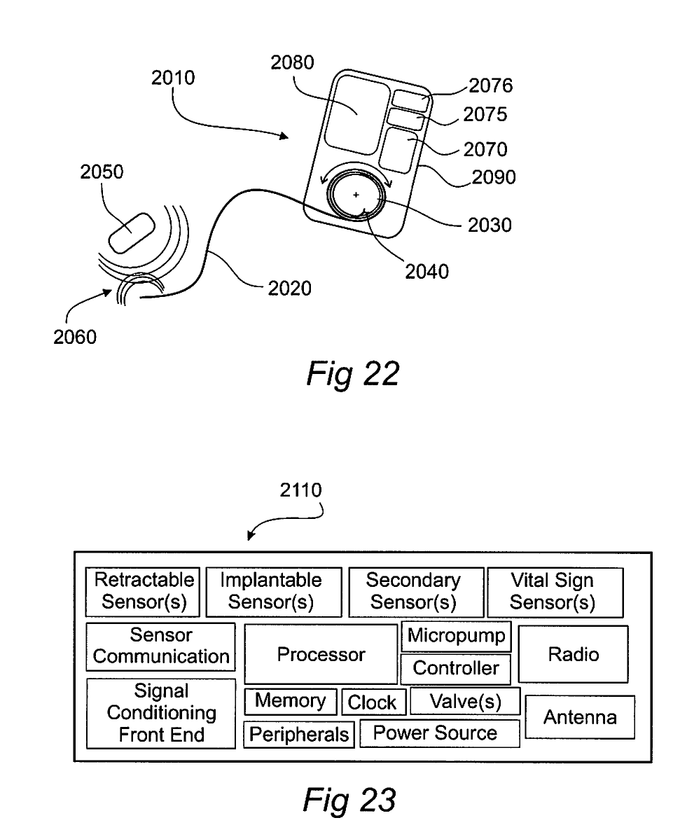

FIG. 22 schematically shows an embodiment of a system for monitoring a surgical site (not explicitly shown) demonstrating an elongate sensing subsystem 2050, an elongate probe 2020 and a communication subsystem 2010 including microcircuitry 2080, a housing 2090, and optionally one or more winding mechanism 2030 optionally having a terminal 2040 for interfacing between the elongate probe 2020 and the communication subsystem 2010. The communication subsystem 2010 may also include one or more micropumps 2070, medicament reservoirs 2075 and sample or waste reservoirs 2076.

The microcircuitry 2080 may include, without limitation, discrete microelectronics, a microcontroller, a microprocessor, a system-on-chip, application specific integrated circuit, a multi-chip mixed signal microcircuit, a field programmable gate array, a field programmable analog array, a digital signal processor, or the like. The microcircuitry 2080 may further include one or more sensors, a sensor front end, an analog to digital converter, a microprocessor, memory, a power source, power management hardware, and the like.

The elongate sensing subsystem 2050 may send or receive signals 2060 to or from the elongate probe 2020 and/or the communication subsystem 2010. The signals 2060 may be optical (e.g. visible light, near infrared light, etc.), electromagnetic (e.g. radio frequency radiation) or acoustic (e.g. ultrasonic waves) in nature. The elongate sensing subsystem 2050 may include one or more emitters and/or receivers to send and/or receive the signals 2060. The elongate probe 2020 may also include one or more emitters and/or receivers to send and/or receive the signals 2060.

The communication subsystem 2010 may include a micropump 2070 and one or more reservoirs 2075, 2076 in fluid communication with the micropump 2070. The elongate probe 2020 may further include a conduit arranged along the length of the elongate probe 2020, the conduit adapted so as to provide fluid communication between the micropump 2070, the reservoir 2075, 2076 and/or the surgical site. The conduit may be arranged down the center line of the elongate probe 2020, or it may be part of a multilumen elongate probe 2020, whereby one or more lumens may provide bidirectional fluid communication between the reservoir 2075, 2076, the micropump 2070 and/or the surgical site. In the case of a multilumen elongate probe 2020, one or more lumens may provide fluid communication from the micropump 2070 and/or reservoir 2075, 2076 to the surgical site. One or more lumens of a multilumen elongate probe 2020 may provide fluid communication from the surgical site to the micropump 2070 and/or reservoir 2075, 2076

The micropump 2070 may be an electromagnetic micropump such as a rotary pump, peristaltic micropump, a syringe pump, or a solenoid driven pump, an active material micropump such as an electroactive polymer micropump, a shape memory material micropump, a piezoelectric micropump, or a piezoceramic micropump.

The micropump 2070 may be adapted to retrieve a fluid sample from the surgical site and deliver the fluid sample to the reservoir 2076. The reservoir 2076 may be a built-in reservoir (e.g. integrally formed), a disposable attachable reservoir, a syringe, or the like. Additionally or alternatively the micropump 2070 may be adapted to deliver a medicament from the reservoir 2075 to the surgical site via the elongate probe 2020. The reservoir 2075 may contain a medicament selected from the group consisting of antibiotics, anti-inflammatory agents, topical anaesthesia, antithrombogenic agents, and thromogenic agents.

The micropump 2070 may be configured to aspirate the surgical site. Aspiration may be valuable during withdrawal of the elongate probe 2020 after an adequate period of monitoring. Alternatively or additionally, in the case of a heavily exuding surgical site, aspiration may be used to deliver exudates to the reservoir 2075 for disposal or testing.

Some suitable medicaments may include, without limitation, topical wound cleansers and antiseptics, antiseptics, hydrogen peroxide, povidone iodine, chlorhexidine diacetate, sodium hypochlorite, antibiotics, bacitracin, polymyxin B, neomycin, silver sulfadiazine, nitrofurazone ointment, gentamicin sulfate, cefazolin, granulation tissue suppressing agents, corticosteroids, activated macrophage supernatant, aloe vera, comfrey (symphytum officinale), eucalyptus, hydrastis canadensis, melaleuca, alternifolia, thymol, trypsin, elase, Granulex™, honey, Lanolin, phenytoin, galium nitrate, gentian violet, recombinant vasoactive protein, scarlet oil, aminoplex,sugardine, tripeptide-copper complex, vitamin E, and the like.

FIG. 23 shows a block diagram of a general layout of the electromechanical components of an embodiment of a communication subsystem 2110. The communication subsystem 2110 includes a retractable sensor, a non-limiting example of which may be an elongate probe, an implantable sensor which may be an element that is placed under the skin as part of the communication subsystem 2110, a sensor communication module, a signal conditioning front end, a processor, memory, peripherals, a clock, one or more secondary sensors, one or more vital sign sensors, one or more micropumps, a controller, one or more valves, a radio, an antenna, and/or power management electronics.

The communication subsystem 2110 may include an alerting component adapted to raise an alarm condition based on the physiological data. The alerting component may include an audible alarm, a visual alarm, a display, a vibratory alarm, a text messaging system, a pager alert, or the like.

The communication subsystem 2110 may include one or more secondary or vital sign sensors selected from the group consisting of an accelerometer, a piezoelectric sensor, a gyroscope, a pressure sensor, an EKG sensor, an EMG sensor, a temperature sensor, a pH sensor, a glucose sensor, and an acoustic sensor.

The communication subsystem 2110 may be a network hub, a local network node, or the like, capable of establishing long range communication between the system and a data network. Alternatively or additionally, the communication subsystem 2110 may be a monitor, capable of communicating information to a user, patient, clinician and/or doctor about the state of the surgical site. The communication subsystem 2110 may also be a mobile computing device such as a mobile phone, an e-reader, a tablet, a media player, a mobile network node, or a pager. The communication subsystem 2110 may also be a hub for establishing communication and transmitting physiologically relevant data between the elongate sensory subsystem and a data network such as an institutional data network, a LAN, a WAN, a cellular network, or the like.

The communication subsystem 2110 may include a combination of an attachment for interfacing with the sensors and a mobile computing device to which it is attached. In this embodiment, the components shown in the block diagram may be shared between the attachment and the mobile phone.

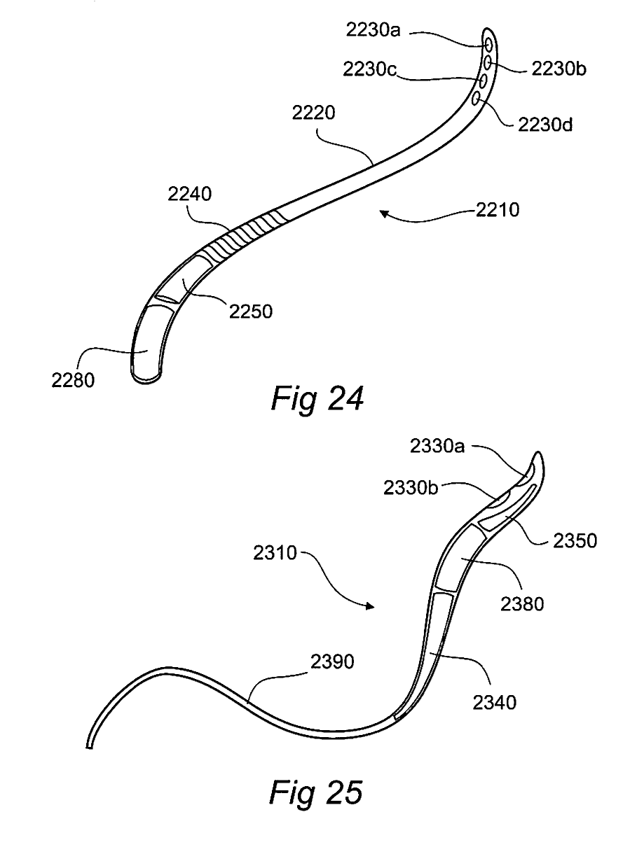

FIG. 24 shows a schematic of a fully implantable embodiment of a sensory subsystem 2210 in accordance with the present disclosure for placement externally to an organ (not explicitly shown). The sensory subsystem 2210 includes an array of sensor elements 2230a-d adapted to interface with a surgical site, organ, transplanted organ, or graft, or an interface between an implant and the surrounding tissue. The sensory subsystem further includes a microcircuit 2250, an antenna 2240 and optionally a power source 2280. The sensory subsystem 2210 further includes an elongate housing that interconnects and provides isolation between the components of the sensory subsystem 2210 and the body.

In this non-limiting example, the antenna 2240 is shown as a helical antenna arranged along at least a portion of the length of the sensory subsystem 2210. The helical antenna 2240 shown provides for additional flexibility and optional stretchable movements perpendicular to and along the length of the sensory subsystem 2210. In another non-limiting example, the antenna 2240 may be a chip antenna, trace antenna, spiral antenna, or the like, embedded within the sensory subsystem 2210.

The sensory elements 2230a-d may be similar to or chosen from those outlined above. In particular, the sensory elements 2230a-d may provide light emitting sources and photodiodes configured to detect changes in local blood oxygen saturation of the associated organ, graft, transplant, or tissue-implant interface.

The microcircuit 2250 is shown in electrical communication with the sensor elements 2230a-d and may be configured to monitor, measure or detect one or more disease states associated with the surgical site including, without limitation, ischemia, phagocytosis related to necrotic tissue consumption, inflammation, apoptosis, progression of wound healing, abscess formation, edema, a tear, and/or onset and/or changes in blood perfusion thereto. Furthermore, the microcircuit 2250 in communication with the sensors 2230a-d may be configured to detect the presence of or measure concentrations of an analyte in or near to the surgical site or organ, transplant, or the like including hemoglobin, lactases, glycerin, water, oxygen, and/or matrix metalloproteinases.

The microcircuit 2250 may include a discrete microcircuit, a microcontroller, a microprocessor, a system-on-chip, application specific integrated circuit, a multi-chip mixed signal microcircuit, a field programmable gate array, a field programmable analog array, a digital signal processor, or the like. The microcircuit 2250 may include sensors, a sensor front end, an analog to digital converter, a microprocessor, memory, a power source, power management hardware, flexible interconnects, or the like.

The optional power source 2280 may include a microbattery or rechargeable microbattery. In addition, the power source 2280 may be a micro fuel cell, a radiation based battery technology, or the like. Alternatively or additionally, the sensory subsystem 2210 may obtain power from an external source. In this case, power may be provided wirelessly by an external RF power source. To facilitate accepting power from an external source, the elongate sensing subsystem 2210 may use an antenna, such as the antenna 2240 shown, for coupling with the RF power source as well as have power management hardware to provide usable power from the harvested signals. In some embodiments, power management may be integrated into the microcircuit 2250. Additionally or alternatively, the sensing subsystem 2210 may be powered via conduction through the body by an externally applied source. The sensing subsystem 2210 may additionally or alternatively be powered via an energy harvesting device, which may harvest ambient RF energy, thermal energy, and/or mechanical energy from the surroundings.

FIG. 25 shows a schematic of an embodiment of a sensory subsystem 2310 in accordance with the present disclosure attached to a withdrawal member 2390. The sensory subsystem 2310 may be similar or equivalent to any embodiment described herein. In the particular embodiment shown, the elongate sensory subsystem 2310 includes a sensing element 2330a-b, a microcircuit 2350 connected to the sensing elements 2330a-b, an optional power source 2380, and an optional antenna 2340. The sensory subsystem 2310 is connected to the withdrawal member 2390. The withdrawal member 2390 extends from the sensory subsystem 2310 to the exterior of the body. Thus, the withdrawal member 2390 provides a means for removing the sensory subsystem 2310 from the body after completion of the monitoring process.

The withdrawal member 2390 may be formed from e.g., a cord, a braid, a fiber, or a multifilament thereof. The withdrawal member may be formed from one or more polymers, metals, carbon fibers, or the like. In general the withdrawal member may be coated with a biocompatible coating and/or a low friction or lubricious coating adapted to minimize adhesion of the withdrawal member to the body during implantation.

The withdrawal member 2390 may be at least partially encapsulated with an elastomeric or gel coating selected from a group consisting of an anti-thrombogenic coating, a non-fouling coating and an anti-bioadhesive coating. Non-limiting examples of suitable polymers may include slow ion release polymers capable of releasing silver ions, carbon, platinum, silver sulfadiazine, chlorhexidine, cadexomer iodine, chlorhexidine gluconate, polyhexamethylene biguanide, antimicrobial peptides, and graphene. Such materials may be provided by Semprus Biosciences, Hydromer, Johnson and Johnson, Arrow International, Cook Critical Care, DSM Biomedical and Edwards Lifesciences among others. Seprafilm™ (Genzyme Corporation) may additionally or alternatively be utilized for an anti-bioadhesive coating.

The withdrawal member 2390 may be suitably applied to several of the embodiments outlined above. In these cases, the withdrawal member 2390 may provide a convenient means for removing the attached sensory subsystem from the body after completion of the monitoring process.

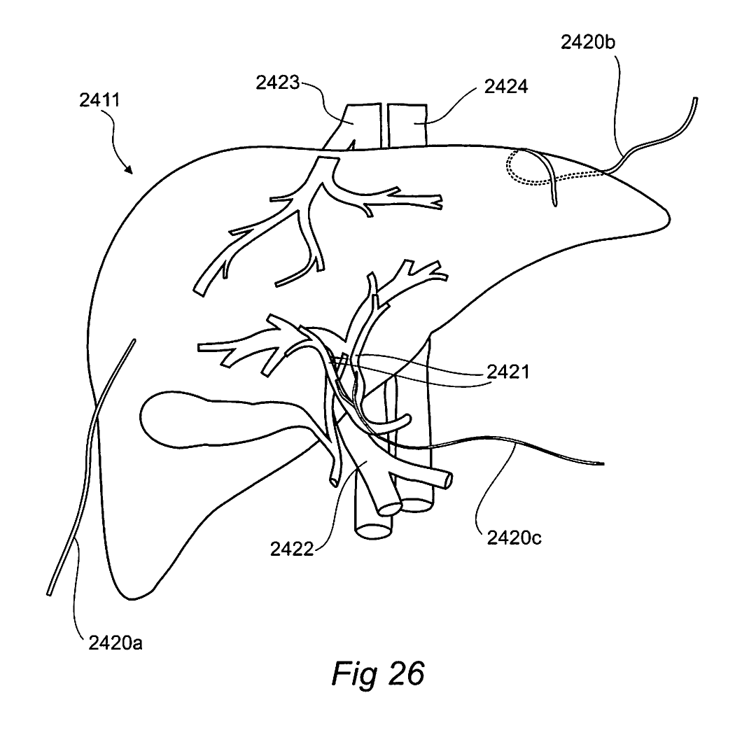

FIG. 26 shows an arrangement of sensory subsystems 2420a-c configured so as to monitor a liver transplant 2411. In addition to the liver transplant 2411 is shown the vascular supply thereto. The left and right hepatic arteries 2421, the portal vein 2422, the hepatic vein 2423, and the aorta 2424 are also shown. A sensory subsystem 2420a is shown adjacent to the right lobe of the liver 2411. A sensory subsystem 2420b is shown adjacent to the left lobe of the liver 2411. A sensory subsystem 2420c is shown attached to the right and left hepatic arteries 2421. Each of the shown sensory subsystems 2420a-c may be placed on the liver 2411 or its vascular supply 2421, 2422, 2423, or 2424 before, during or after the implantation procedure. The sensory subsystems 2420a-c may be attached as described herein and may be attached using a temporary attachment method such as e.g. a degradable adhesive. The sensory subsystems 2420a-c may be positioned on the surface of the liver 2411 or may be adapted so as to be slightly inserted into the tissues of the liver 2411.

The sensory subsystems 2420a-c may be arranged with sensors and components as outlined above and in previously discussed embodiments. In particular, the sensory subsystems 2420a-b may be adapted to monitor partial pressure of oxygen, oxygen saturation, edema buildup, or the like in adjacent tissues of the liver 2411. Alternatively or additionally, the sensory subsystem 2420c may be configured so as to monitor blood flow through the right and/or left hepatic arteries 2421. If blood flow to either the right or left hepatic arteries 2421 is compromised, the transplant can be compromised.

In order to monitor the blood flow through the right and/or left hepatic arteries 2421, the sensory subsystem 2420c may include an energy source and a detector situated so as to provide and detect energy to and from the left and/or right hepatic arteries 2421. The energy source may be an audible, ultrasonic, microwave, infrared, or visible source, and the detector may be adapted as necessary to detect the associated energy reflected from or passing through the left and/or right hepatic arteries 2421. In particular, the sensory subsystem 2420c may include one or more narrow band or wideband light sources in combination with one or more narrow band or wideband photodetectors. The sensory subsystem 2420c may thus monitor changes in the absorption, reflectance, or dispersion of light through the left and/or right hepatic arteries over time. Changes in these signals can relate to changes in the oxygen partial pressure, oxygen saturation within or blood flow through the left and/or right hepatic arteries 2421.

Although a liver transplant 2411 is shown, the organ may be a colon, small intestine, bile duct, pancreas, stomach, esophagus, kidney, lung, artery, vein, ureter, urinary bladder, urethra, nerve or portion thereof. Additionally the organ may be a tissue engineered organ, graft or the like.

The sensory subsystems 2420a-c may further include sensors and associated microelectronics so as to monitor oxygen perfusion, local anatomy changes, bioimpedance changes, or bioimpedance tomography changes within the organ, transplant, near to the tissue/implant interface, and/or within the vascular supply thereto.

The sensory subsystems 2420a-c may be attached to one or more withdrawal members, adapted so as to connect the associated sensory subsystem 2420a-c with the outside of the body. The withdrawal member may be formed from a cord, a braid, a fiber or multifilament thereof. The withdrawal member may be formed from one or more polymers, metals, carbon fibers, or the like. The withdrawal member may be coated with a biocompatible coating and/or a low friction or lubricious coating to minimize adhesion of the withdrawal member to the body during implantation.

Generally speaking, the sensory subsystem may be adapted to monitor the tissue viability of the transplanted organ, tissue engineered construct, graft or tissue/implant interface. Additionally or alternatively, the sensory subsystem may be adapted to monitor the health of the vascular supply to a transplanted organ, tissue engineered construct, graft or tissue/implant interface prior to, during and/or after completion of a surgical procedure. The sensory subsystem may be further adapted to monitor blood flow through the vascular supply prior to, during and/or after completion of a surgical procedure. In particular, the sensory subsystem may be adapted to monitor the patency of the right and/or left hepatic arteries in the vicinity of a liver.

The sensory subsystem may be sutured, stapled, glued or placed to the organ or tissue engineered construct prior to, during or after completion of the surgical procedure. The sensory subsystem may further be embedded into an implant so as to monitor the tissue/implant interface after a surgical procedure.

Suitable organs may be selected from a group consisting of a liver, colon, small intestine, bile duct, pancreas, stomach, esophagus, kidney, lung, artery, vein, ureter, urinary bladder, urethra, and nerve.

Tissue engineered constructs and vessels may be constructed and precursor materials selected from a range of prior art. The tissue engineered construct may be fabricated from a range of cell sources including endothelial cells (EC), vascular smooth muscle cells (SMCs), fibroblasts, myofibroblasts, stem cells, and/or pericytes. In the case of a blood vessel construct including an adventitia, a contractile media, and an intima, the construct may be produced using cell based tissue engineering methods. Vascular constructs can be produced from dermal fibroblasts, saphenous vein fibroblasts, and/or vascular SMCs. Relating to fabrication using tissue engineering methods, the tissue constructs may be fabricated in sheets to form adherent living tissue sheets. The sheets may then be rolled onto a tubular support and further cultured to form a tissue engineered vascular graft. A sensory subsystem may be incorporated into the tissue engineered construct at any stage in the fabrication process. In particular, a sensory subsystem may be incorporated between sheets during the rolling step of the fabrication process.

Tissue engineered constructs and vessels may be constructed from any suitable precursor materials.

The sensory subsystem may be directly attached to or embedded into a tissue engineered construct or vessel prior to implantation and/or even during the fabrication or growth process thereof.

Another embodiment of a sensory subsystem is disclosed herein that may be adapted for monitoring the patency of a stent. The disclosed sensory subsystem may be attached to the stent prior to, during or after placement of the stent. The sensory subsystem may be adapted to monitor flow of fluid through the stent during or after placement. Alternatively, additionally, or in combination, the sensory subsystem may be adapted to monitor tissue viability and/or anatomical changes in and around the stent during or after placement.

The stent may be selected from a group consisting of a vascular, ureteral, biliary, pancreatic, esophageal, bronchial and tracheal stent.

A method for monitoring a surgical site within a body is disclosed herein. The method includes placement of a sensory subsystem prior to, during or after completion of a surgical procedure, monitoring the surgical site with the sensory subsystem for a period of hours, days, weeks, or months, and removing the sensory subsystem from the body.

Yet another method for monitoring a surgical site within a body is disclosed herein. The method includes placement of a sensory subsystem and attached withdrawal member prior to, during or after a surgical procedure, the sensory subsystem is placed to interface with the surgical site, and the withdrawal member placed so as to span from the sensory subsystem to the exterior of the body. The method further includes monitoring the surgical site with the sensory subsystem for a period of hours, days, weeks or months, and removing the sensory subsystem from the body by pulling out the withdrawal member.

It will be appreciated that additional advantages and modifications will readily occur to those skilled in the art. Therefore, the disclosures presented herein and broader aspects thereof are not limited to the specific details and representative embodiments shown and described herein. Accordingly, many modifications, equivalents, and improvements may be included without departing from the spirit or scope of the general inventive concept as defined by the appended claims and their equivalents.

What is claimed is:

1-39. (canceled)

40. A system for monitoring a site within a mammalian body, comprising:
a sensing subsystem adapted for placement within the mammalian body, comprising:
a substrate;
at least one sensor disposed on the substrate and adapted to sense a physiological parameter of the mammalian body; and
a microcircuit in operable communication with the at least one sensor and configured to communicate with a communication subsystem; and
a communication subsystem in operable communication with the sensing subsystem.

41. The system in accordance with claim 40, wherein the substrate is formed from elastomeric material.

42. The system in accordance with claim 41, wherein the elastomeric substrate is formed from material selected from the group consisting of a silicone elastomer and an elastic protein.

43. The system in accordance claim 41, wherein the elastomeric substrate is formed of a material selected from the group consisting of a poly(dimethylsiloxane), a viscoelastic gel, a collagen, a porous core elastomer, a perfluoropolyether, a silicone-containing polyurethane, a polyurethane, a PFPE-PDMS block copolymers, a polyisoprene, a polybutadiene, a fluoroolefin-based fluoroelastomers, and a resilin.

44. The system in accordance with claim 41, wherein the microcircuit is operably coupled to the at least one sensor by a flexible interconnect having the capability to stretch relative to its non-stretched length, without damage by an amount selected from the group consisting of up to 10% stretch, up to 30% stretch, up to 50% stretch, and up to 100% stretch.

45. The system in accordance with claim 40, wherein the sensing subsystem includes one or more bioadhesives configured to anchor the sensing subsystem to a body tissue.

46. The system in accordance with claim 45, wherein the sensing subsystem further comprises a biodegradable bioadhesive.

47. The system in accordance with claim 40, wherein the at least one sensor includes an elastomeric capacitive strain sensor.

48. The system in accordance with claim 40, wherein the sensing subsystem is configured and dimensioned for placement across a surgical site.

49. The system in accordance with claim 48, wherein the sensing subsystem is configured and dimensioned to sense a biological property selected from the group consisting of a bioelectric activity, a biopotential, a bioimpedance, a bioimpedance tomography, a motility, a water content, an ischemia, and a tissue oxygenation.

50. The system in accordance claim 48, wherein the sensing subsystem is configured to sense a disease state selected from the group consisting of (appears in claim 10), a phagocytosis, an inflammation, an apoptosis, a progression of wound healing, an abscess, an edema, a tear, and a leak.

51. The system in accordance with claim 40, wherein the communication subsystem is configured for attachment to the body.

52. The system in accordance with claim 51, wherein the sensing subsystem and the communication subsystem are in wireless communication.

53. The system in accordance with claim 40, further comprising at least one light source configured to emit light towards a surgical site, at least one of the sensors configured and dimensioned to accept light from the surgical site.

54. The system in accordance with claim 40, wherein the sensing subsystem is configured and dimensioned for placement entirely within an organ of the body, the sensing subsystem and the communication subsystem being in wireless communication.

55. The system in accordance with claim 54, comprising one or more additional sensing subsystems configured for placement outside the organ, each of the additional sensing subsystems in communication with one or more of the sensing subsystems and/or the communication subsystem.

56. The system in accordance with claim 55, wherein one or more of the sensing subsystems is configured and dimensioned to transmit a signal and one or more of the sensing subsystems is configured and dimensioned to receive the signal, the system configured and dimensioned to analyze the received signal to determine an integrity state of the organ.

57. The system in accordance with claim 56, wherein one or more of the sensing subsystems is configured and dimensioned to determine the integrity of an anastomosis.

58. A method for determining an integrity state of a wall of an organ, comprising:
implanting a first device including one or more first sensors within the organ in close proximity to the wall;
positioning one or more additional devices each including one or more additional sensors in close proximity to the organ;
communicating one or more signals between the first device and one or more of the additional devices while recording the received signals with at least one of the first and/or additional sensors; and
analyzing the received signals to determine the integrity of the wall.

59. The method as claimed in claim 58, further comprising monitoring a bioimpedance between the first device and one or more of the additional devices.
